(19) United States
(12) Patent Application Publication
(10) Pub. No.: US 2023/0404989 A1
(43) Pub. Date: Dec. 21, 2023

(54) GRK2 INHIBITION BY PAROXETINE AMELIORATES OSTEOARTHRITIS

(71) Applicant: THE PENN STATE RESEARCH FOUNDATION, University Park, PA (US)

(72) Inventor: Fadia KAMAL, University Park, PA (US)

(21) Appl. No.: 18/036,837

(22) PCT Filed: Nov. 12, 2021
(86) PCT No.: PCT/US2021/059186

§ 371 Date: May 12, 2023

Related U.S. Application Data
(60) Provisional application No. 63/113,021, filed on Nov. 12, 2020.

(51) Int. Cl. A61K 31/4525; A61K 38/29; A61P 19/02

(52) U.S. Cl. CPC A61K 31/4525; A61K 38/29; A61P 19/02

(57) ABSTRACT

The present disclosure is directed to compositions and methods for the treatment of inflammation, particularly methods using compositions containing paroxetine or a pharmaceutically acceptable salt or derivative thereof.

### STATEMENT REGARDING FEDERALLY SPONSORED RESEARCH OR DEVELOPMENT

This invention was made with government support under Grant No. AR071968 awarded by the National Institutes of Health. The Government has certain rights in the invention.

### BACKGROUND

Osteoarthritis (OA) is a debilitating joint disease characterized by progressive cartilage degeneration, with no available disease-modifying therapy. OA is driven by pathological chondrocyte hypertrophy (CH), whose cellular regulators are unknown. CH being the principle cellular process leading to cartilage degeneration. While there is no treatment that can stop cartilage degeneration in OA, parathyroid hormone (PTH) has demonstrated chondroprotective and chondroregenerative effects in mice. PTH works through parathyroid hormone receptor type-1 (PTH1R), a GPCR expressed in growth plate and articular chondrocytes. In growth plate chondrocytes, PTH1R-Gas signaling maintains chondrocyte homeostasis and prevents their hypertrophy. We have previously shown that GPCR GRK2 signaling is elevated in other diseases leading to GPCR desensitization and the loss of Ga signaling, and that inhibition of GRK2 attenuates disease progression. The therapeutic efficacy of G-protein coupled receptor kinase 2 (GRK2) inhibition in other diseases by recovering protective G-protein coupled receptor (GPCR) signaling has been reported. However, the role of GPCR-GRK2 pathway in OA is unknown.

Therefore, there is a need for compositions and methods of treating osteoarthritis. The compositions and methods disclosed herein address these and other needs.

### SUMMARY

Provided herein is a pharmaceutical composition including paroxetine or a pharmaceutically acceptable salt or derivative thereof; and one or more pharmaceutical acceptable carriers. In some embodiments, the compound is present in an effective amount to increase PTH/PTH1R-mediated cAMP production by at least 2 fold. In some embodiments, the PTH/PTH1R-mediated cAMP production is increased by inhibiting G-protein coupled receptor kinase 2 (GRK2). In some embodiments, the composition further includes parathyroid hormone (PTH).

Provided is also a method of treating an inflammatory disorder in a subject in need thereof, the method including administering a therapeutically effective amount of a paroxetine or a pharmaceutically acceptable salt or derivative thereof, or the pharmaceutical composition including paroxetine or a pharmaceutically acceptable salt or derivative thereof to the subject. In some embodiments, the inflammatory disorder is osteoarthritis.

Described herein is also a method of increasing PTH/PTH1R-mediated cAMP production by inhibiting G-protein coupled receptor kinase 2 (GRK2) in a subject in need thereof, the method including administering a therapeutically effective amount of a paroxetine or a pharmaceutically acceptable salt or derivative thereof, or the pharmaceutical composition including a paroxetine or a pharmaceutically acceptable salt or derivative thereof to the subject.

The details of one or more embodiments of the disclosure are set forth in the accompanying drawings and the description below. Other features, objects, and advantages of the disclosure will be apparent from the description and drawings, and from the claims.

### DESCRIPTION OF DRAWINGS

FIG. 1 is a bar graph that shows in vitro cAMP. Paroxetine promotes PTH1R Gas/cAMP signaling in the articular cartilage of DMM mice. cAMP produced in cartilage obtained from sham or DMM mice that were treated with vehicle or paroxetine (Px) for 8 weeks. Mouse cartilage was stimulated with PTH ex vivo and tissue cAMP levels were measured 30 minutes following stimulation. ***P<0.001 & ****P<0.0001. N=5/group.

FIG. 2A-2D are images of (A) Safranin 0-Fast Green staining of cartilage surface. (B) Total tibial uncalcified cartilage areas (TUC), total chondrocyte number and matrix positive chondrocyte numbers of sham mice or DMM mice treated with vehicle (V), paroxetine (Px), PTH, or combination (PTH+Px).

FIG. 3A-3C shows increased GRK2 expression associated with reduced cAMP levels is detected in OA chondrocytes. (A) Timeline for DMM time-course study. (B) Quantification of cAMP level in the whole tibial articular surface isolated from mice described in (A); N=5/group. (C) cAMP levels measured in (B) normalized to the total number of chondrocytes in tibial articular surface. In (B) and (C), cAMP level in 12 weeks sham group is set as 100%; **P<0.01, ***P<0.001 & ****P<0.0001 versus 2 wk sham, and #P<0.05, ^{##}P<0.01, ^{###}<& ^{####}P<0.0001 versus 12 weeks sham using one-way ANOVA. Values are expressed as mean±SEM.

FIG. 4A-4H shows chondrocyte-specific deletion of GRK2 attenuates OA progression. (A) Experimental timeline used to confirm conditional deletion of GRK2 in articular chondrocytes. Control (Ctrl, GRK29 and GRK2-cKO (GRK2^{ff}/Agc1^{tm(IRES-CreERT2)}) mice received DMM surgery followed by tamoxifen injections 7 weeks later, and were sacrificed one week later. (B) Experimental timeline used to investigate the impact of chondrocyte-specific GRK2 deletion on OA progression. Tamoxifen was injected at week 7 to induce GRK2 gene deletion at week 8, and mice were harvested at week 12 post-DMM. (C) OARSI score of cartilage tissues of Safranin-O and Fast Green-stained knee joints harvested from control and GRK2-cKO mice described in B demonstrating the extent of injury 12 weeks post-DMM. (D-F) Histomorphometric analyses of uncalcified cartilage area (D), total number of chondrocytes (E), and number of matrix-producing chondrocytes (F) in tibial articular cartilage of Safranin-O and Fast Green-stained knee joints harvested from control and GRK2-cKO mice described in B (G) Quantification of cAMP level in the whole tibial articular surface isolated from mice described in (B); N=5/group. (H) cAMP levels measured in (G) normalized to the total number of chondrocytes in tibial articular surface. In (G) and (H), cAMP level in Ctrl-sham group was set as 100%; cAMP levels were measured in tibial articular cartilage pooled from three mice per data point. **P<0.01, ***P<0.001 & ****P<0.0001 versus non-KO control mice using unpaired t-test. Values are presented as mean±SEM.

FIG. 5A-5G shows that paroxetine prevents OA progression. (A) Schematic representation of timeline of drug treatment following sham (N=6) or DMM surgeries: 8 weeks following surgery, mice received daily i.p. injection of Vehicle (V, N=5), Paroxetine (Px, N=7), Gallein (G, N=8), Fluoxetine (Fx, N=6), or Indomethacin (Ind, N=7). All mice were harvested 12 weeks following surgery. Green arrows denote the duration of drug treatment. (B) Mouse OARSI score showing the extent of injury in DMM mice receiving different treatments. (C-E) Histomorphometric analyses of all treatment groups showing changes in uncalcified cartilage area (C), total chondrocyte number (D), and matrix-producing chondrocyte number (E). Dashed red line indicates the level of each analyzed parameter 8 weeks post-DMM, i.e., at the time point drug treatment was initiated; N=5. (F) Quantification of cAMP level in the whole tibial articular surface isolated from mice described in (A); N=5/group. (G) cAMP levels measured in (F) normalized to the total number of chondrocytes in tibial articular surface. In (F) and (G), cAMP level in sham group was set as 100%; cAMP levels were measured in tibial articular cartilage pooled from three mice per data point; N=5/group. *P<0.05, ***P<0.001 & ****P<0.0001 versus DMM vehicle control group; ^{##}P<0.01, ^{###}P<0.001, & ^{####}P<0.0001 versus sham; ^{@}P<0.05, ^{@@@}P<0.001, ^{@@@@}P<0.0001 versus 8-weeks DMM; ^{$}P<0.05, ^{$$}P<0.01, ^{$$$}P<0.001 & ^{$$$$}P<0.0001 versus DMM+G group; ^{\hat{ }}P<0.05, ^{\hat{ }\hat{ }\hat{ }}P<0.001, ^{\hat{ }\hat{ }\hat{ }\hat{ }}P<0.0001 versus DMM+Px group using one-way ANOVA. Values are expressed as mean±SEM.

FIG. 6A-6F shows that paroxetine treatment but not inducible chondrocyte-specific GRK2 deletion attenuates synovitis after DMM. (A) Representative images of Safranin-O and Fast Green staining of the anterior femoral synovial region of mouse knee sections 12 weeks after DMM or sham surgery, mice received daily drug treatment following the timeline described in FIG. 5B; scale bar=50 μm. (B) Magnified images of regions indicated in red boxes in (A); scale bar=10 μm. (C) Quantification of anterior femoral synovial membrane thickness in sham (N=6) and DMM mice receiving treatment with vehicle (V; N=5), paroxetine (Px; N=7), gallein (G; N=8), fluoxetine (Fx; N=6) or indomethacin (Indo; N=7). Dashed red line indicates levels of defined parameters in 8 weeks post-DMM mice, prior to drug treatment initiation. (D) Representative images of Safranin-O and Fast Green staining of anterior femoral synovium of mouse knee sections in non-knockout control (ctrl; N=6) and GRK2 conditional knockout (GRK2 c-KO; N=5) mice 12 weeks after DMM surgery, study timeline described in FIG. 4C; scale bar=50 μm. (E) Magnified images of regions indicated in red boxes in (D); scale bar=10 μm. (F) Quantification of synovial thickness in control and GRK2-cKO mice. Yellow line depicts synovial lining thickness. ^{####}P<0.0001 vs sham; **P<0.01 and ****P<0.0001 vs DMM+V; ^{@@@@}P<0.0001 versus 8-weeks DMM using one-way ANOVA. Values are expressed as mean±SEM.

FIG. 7A-7D shows that paroxetine normalizes chondrocyte GRK2 expression and cAMP levels, and attenuates chondrocyte hypertrophy in human OA cartilage. Osteochondral plugs obtained from each patient were cultured ex vivo and treated with vehicle (V), gallein (G) or paroxetine (Px) for 48 hours. GRK2, ADAMTS5, and MMP13 IF staining images were taken. (A) Quantification of human GRK2 gene expression normalized to GAPDH by RT-qPCR. (B) Percent change in cAMP levels vs vehicle treated group. Quantification of human (C) ADAMTS5 and (D) MMP13 gene expression normalized to GAPDH by RT-qPCR. *P<0.05 and **P<0.01 vs the vehicle treated group using paired student t-test, values are expressed as mean±SEM; N=5.

FIG. 8A-8I shows that paroxetine inhibits joint bone remodeling in DMM mice. Microstructural assessment of subchondral bone changes in mice following drug treatment study timeline, described in FIG. 5C, using Micro-Computed Tomography (Micro-CT). (A) 5D reconstructed images of knee joints highlighting changes to the femoral and tibial surfaces as well as the medial and lateral menisci. (D) Sagittal view of the medial joint compartment visualizing subchondral bone changes, a black line marks subchondral plate thickness. Quantified changes in (B) knee bone volume (BV), (C) knee total volume (TV), (E) subchondral bone plate thickness, (F) subchondral bone mineral density (BMD), (G) subchondral bone volume to total volume ratio (BV/TV), (H) subchondral trabecular thickness (Tb.Th), and (I) number of osteophytes. Each bar represents mean±SEM. Sham age matched wild type normal mice (N=5); DMM mice treated with vehicle (V; N=5), paroxetine (Px; N=7), or gallein (G; N=8). ^{#}P<0.05, ^{##}P<0.01 and ^{####}P<0.0001 vs. Sham; *P<0.05, **P<0.01, ***P<0.001 and ****P<0.0001 vs. DMM+V using one-way ANOVA. ^{&}P<0.05 vs. sham & ^{$}P<0.05 vs. DMM+V using unpaired t-test.

FIG. 9 shows GRK2 inhibition as a novel therapeutic approach for OA. (A) Illustration of the knee joint showing phenotypical differences between normal and OA joints, including articular cartilage degeneration and increased chondrocyte hypertrophy, subchondral bone sclerosis, and synovial thickening. (B) Ligand binding to GPCRs on the chondrocyte plasma membrane activates Gas and Gβγ subunits. Gas activates adenylyl cyclase (AC) leading to increased cAMP levels, which maintain chondrocyte anabolic signaling and cartilage homeostasis. GRK2 is recruited by Gβγ to phosphorylate the receptor for internalization and signal termination. (C) In OA chondrocytes, intracellular GRK2 is upregulated, desensitizing the GPCRs and reducing Gas activation and cAMP production, leading to chondrocyte hypertrophy and their shift to a catabolic phenotype, which plays a central role in cartilage degeneration, a hallmark of OA. (D) Illustration of Gβγ-GRK2 inhibition as a novel therapeutic approach in OA. (D1) The GRK2 inhibiting SSRI paroxetine (Px), and (D2) the novel Gβγ inhibitor gallein (G), inhibit GRK2 recruitment to the cell membrane and the subsequent GPCR desensitization, thus recovering the impaired Gas-cAMP signaling. Chondrocyte anabolic signaling and cartilage homeostasis are restored due to reactivated Gas signaling and rising cAMP levels.

FIG. 10A-10E shows histomorphometric changes in DMM mice during the time-course of OA development. (A) OA development experimental timeline. Mice received sham (N=6) or DMM surgery then were harvested 8 weeks (N=5) or 12 weeks (N=5) following surgery. Mice demonstrated a time dependent increase in (B) OARSI score with corresponding decreases in (C) uncalcified cartilage area, (D) Total chondrocyte number and (E) matrix-producing chondrocyte number. Eight weeks following DMM represents clinically progressive OA that is not yet end stage OA, as displayed by mild cartilage degeneration characterized by significant chondrocyte loss and hypertrophy. ^{##}P<0.01, ^{###}P<0.001, & ^{####}P<0.0001 versus sham; *P<0.05 & ****P<0.0001 versus 12 weeks post-DMM group using one-way ANOVA, values are expressed as mean±SEM.

FIG. 11A-11D shows that short term chondrocyte-specific GRK2 deletion or pharmacologic inhibition recovers Gas signaling in DMM mice. (A) Short term GRK2 conditional KO study timeline. Tamoxifen (Tam) was injected on week seven to induce chondrocyte GRK2 deletion by week 8, and mice were harvested at 9 weeks post-surgery. (B) Analysis of change in cAMP production in control versus GRK2-cKO mice receiving sham or DMM surgery. ***P<0.001 and ****P<0.0001 using two-way ANOVA. (C) Short term drug treatment study timeline. Mice received drug treatments for one week beginning 8 weeks post-DMM and were harvested at 9 weeks post-DMM. (D) Change in cAMP production in DMM mice vs sham mice following 1 week of treatment with vehicle, paroxetine (Px), gallein (G), fluoxetine(Fx), or indomethacin (Indo). cAMP levels were measured in tibial articular cartilage pooled from three mice per data point; N=5/group. ***P<0.001, ****P<0.0001 versus DMM+V; ^{#}P<0.05, ^{##}P<0.01, & ^{####}P<0.0001 versus sham; ^{$$$}P<0.001 versus DMM+G; and ^{\hat{ }\hat{ }\hat{ }}P<0.001 versus DMM+Px using one-way ANOVA, values are expressed as mean±SEM.

FIG. 12A-12H shows that inducible conditional GRK2 deletion in sham operated knees promotes their chondrocyte anabolic activity. (A) Chondrocyte specific GRK2 deletion study timeline with tamoxifen injections initiated 7 weeks post-DMM to induce GRK2 deletion 8 weeks post-DMM, mice were harvested 12 weeks post-DMM. (B) Representative 10×images of Safranin-O and Fast Green stained knee joints, red box indicates the region magnified (20×) and displayed in (D). (C-G) Histomorphometric analyses of uncalcified cartilage area, total chondrocyte number, matrix producing chondrocyte number, and matrix non-producing chondrocyte number in control (ctrl) versus GRK2-cKO mice; N=6/group. (H) Analysis of cAMP levels in ctrl versus GRK2-cKO mice. cAMP levels were measured in tibial articular cartilage pooled from three mice per data point; N=5/group. **P<0.01 & ****P<0.0001 versus non-KO control mice using Welch's t-test, values are expressed as mean±SEM.

FIG. 13A-13C shows that paroxetine in combination with low dose of PTH prevents OA progression. 8 weeks following surgery, mice received daily i.p. injection of Vehicle (V, N=6), recombinant human Parathyroid hormone (PTH, N=6), or a combination of recombinant human Parathyroid hormone and Paroxetine (Px, N=9). Sham-operated mice (N=5) were used as the non-OA control. All mice were harvested 12 weeks following surgery. (13A-13C) Histomorphometric analyses of all treatment groups showing changes in uncalcified cartilage area (13A), total chondrocyte number (13B), and matrix-producing chondrocyte number (13C). *P<0.05, **P<0.01, ***P<0.001 & ****P<0.0001 using one-way ANOVA. Values are expressed as mean±SEM.

FIG. 14A-14E shows that paroxetine inhibits the deleterious effects of PTH on the subchondral bone of OA mice. Microstructural assessment of subchondral bone changes in mice following the experiment described in FIG. 13, using Micro-Computed Tomography (Micro-CT). Quantified changes in subchondral bone (14A) plate thickness, (14B) bone mineral density (BMD), (14C) bone volume to total volume ratio (BV/TV), (14D) trabecular thickness (Tb.Th), and (14E) number of osteophytes. Sham age matched wild type normal mice (N=6); DMM mice treated with Vehicle (V, N=6), recombinant human Parathyroid hormone (PTH, N=6), or a combination of recombinant human Parathyroid hormone and Paroxetine (Px, N=9). *P<0.05, **P<0.01, & ****P<0.0001 using one-way ANOVA. Values are expressed as mean±SEM.

FIG. 15. is a bar graph that shows in vivo changes in pain. Paroxetine attenuates pain sensitivity of DMM mice. Response threshold to mechanical pain stimulus measured in sham mice and DMM mice 12 weeks following DMM. DMM mice received respective daily treatments from week 8 until week 12 following DMM: vehicle (V) or Paroxetine (Px). *P<0.05 & ***P<0.001.

FIG. 16. is a bar graph that shows in vivo changes in gait. Paroxetine normalizes changes in the gait of DMM mice. Stride length in centimeters measured for sham mice and DMM mice 12 weeks following DMM. DMM mice received respective daily treatments from week 8 until week 12 following DMM: vehicle (V) or Paroxetine (Px). **P<0.01 & ***P<0.001

Like reference symbols in the various drawings indicate like elements.

### DETAILED DESCRIPTION

A number of embodiments of the disclosure have been described. Nevertheless, it will be understood that various modifications may be made without departing from the spirit and scope of the invention.

### Definitions

To facilitate understanding of the disclosure set forth herein, a number of terms are defined below. Unless defined otherwise, all technical and scientific terms used herein generally have the same meaning as commonly understood by one of ordinary skill in the art to which this disclosure belongs.

### General Definitions

To facilitate understanding of the disclosure set forth herein, a number of terms are defined below. Unless defined otherwise, all technical and scientific terms used herein have the same meanings as commonly understood by one of skill in the art to which the disclosed invention belongs. Publications cited herein and the materials for which they are cited are specifically incorporated by reference.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than where noted, all numbers expressing quantities of ingredients, reaction conditions, geometries, dimensions, and so forth used in the specification and claims are to be understood at the very least, and not as an attempt to limit the application of the doctrine of equivalents to the scope of the claims, to be construed in light of the number of significant digits and ordinary rounding approaches.

As used in this specification and the following claims, the terms “comprise” (as well as forms, derivatives, or variations thereof, such as “comprising” and “comprises”) and “include” (as well as forms, derivatives, or variations thereof, such as “including” and “includes”) are inclusive (i.e., open-ended) and do not exclude additional elements or steps. For example, the terms “comprise” and/or “comprising,” when used in this specification, specify the presence of stated features, integers, steps, operations, elements, and/or components, but do not preclude the presence or addition of one or more other features, integers, steps, operations, elements, components, and/or groups thereof. Accordingly, these terms are intended to not only cover the recited element(s) or step(s), but may also include other elements or steps not expressly recited. Furthermore, as used herein, the use of the terms “a”, “an”, and “the” when used in conjunction with an element may mean “one,” but it is also consistent with the meaning of “one or more,” “at least one,” and “one or more than one.” Therefore, an element preceded by “a” or “an” does not, without more constraints, preclude the existence of additional identical elements.

The use of the term “about” applies to all numeric values, whether or not explicitly indicated. This term generally refers to a range of numbers that one of ordinary skill in the art would consider as a reasonable amount of deviation to the recited numeric values (i.e., having the equivalent function or result). For example, this term can be construed as including a deviation of +10 percent of the given numeric value provided such a deviation does not alter the end function or result of the value. Therefore, a value of about 1% can be construed to be a range from 0.9% to 1.1%. Furthermore, a range may be construed to include the start and the end of the range. For example, a range of 10% to 20% (i.e., range of 10%-20%) can includes 10% and also includes 20%, and includes percentages in between 10% and 20%, unless explicitly stated otherwise herein.

It is understood that when combinations, subsets, groups, etc. of elements are disclosed (e.g., combinations of components in a composition, or combinations of steps in a method), that while specific reference of each of the various individual and collective combinations and permutations of these elements may not be explicitly disclosed, each is specifically contemplated and described herein. By way of example, if a composition is described herein as including a component of type A, a component of type B, a component of type C, or any combination thereof, it is understood that this phrase describes all of the various individual and collective combinations and permutations of these components. For example, in some embodiments, the composition described by this phrase could include only a component of type A. In some embodiments, the composition described by this phrase could include only a component of type B. In some embodiments, the composition described by this phrase could include only a component of type C. In some embodiments, the composition described by this phrase could include a component of type A and a component of type B. In some embodiments, the composition described by this phrase could include a component of type A and a component of type C. In some embodiments, the composition described by this phrase could include a component of type B and a component of type C. In some embodiments, the composition described by this phrase could include a component of type A, a component of type B, and a component of type C. In some embodiments, the composition described by this phrase could include two or more components of type A (e.g., A1 and A2). In some embodiments, the composition described by this phrase could include two or more components of type B (e.g., B1 and B2). In some embodiments, the composition described by this phrase could include two or more components of type C (e.g., C1 and C2). In some embodiments, the composition described by this phrase could include two or more of a first component (e.g., two or more components of type A (A1 and A2)), optionally one or more of a second component (e.g., optionally one or more components of type B), and optionally one or more of a third component (e.g., optionally one or more components of type C). In some embodiments, the composition described by this phrase could include two or more of a first component (e.g., two or more components of type B (B1 and B2)), optionally one or more of a second component (e.g., optionally one or more components of type A), and optionally one or more of a third component (e.g., optionally one or more components of type C). In some embodiments, the composition described by this phrase could include two or more of a first component (e.g., two or more components of type C (C1 and C2)), optionally one or more of a second component (e.g., optionally one or more components of type A), and optionally one or more of a third component (e.g., optionally one or more components of type B).

Ranges can be expressed herein as from “about” one particular value, and/or to “about” another particular value. By “about” is meant within 5% of the value, e.g., within 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

Administration” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like. “Concurrent administration”, “administration in combination”, “simultaneous administration” or “administered simultaneously” as used herein, means that the compounds are administered at the same point in time or essentially immediately following one another. In the latter case, the two compounds are administered at times sufficiently close that the results observed are indistinguishable from those achieved when the compounds are administered at the same point in time. “Systemic administration” refers to the introducing or delivering to a subject an agent via a route which introduces or delivers the agent to extensive areas of the subject's body (e.g. greater than 50% of the body), for example through entrance into the circulatory or lymph systems. By contrast, “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration but are undetectable or detectable at negligible amounts in distal parts of the subject's body. Administration includes self-administration and the administration by another.

As used here, the terms “beneficial agent” and “active agent” are used interchangeably herein to refer to a chemical compound or composition that has a beneficial biological effect. Beneficial biological effects include both therapeutic effects, i.e., treatment of a disorder or other undesirable physiological condition, and prophylactic effects, i.e., prevention of a disorder or other undesirable physiological condition. The terms also encompass pharmaceutically acceptable, pharmacologically active derivatives of beneficial agents specifically mentioned herein, including, but not limited to, salts, esters, amides, prodrugs, active metabolites, isomers, fragments, analogs, and the like. When the terms “beneficial agent” or “active agent” are used, then, or when a particular agent is specifically identified, it is to be understood that the term includes the agent per se as well as pharmaceutically acceptable, pharmacologically active salts, esters, amides, prodrugs, conjugates, active metabolites, isomers, fragments, analogs, etc.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

“Inactivate”, “inactivating” and “inactivation” means to decrease or eliminate an activity, response, condition, disease, or other biological parameter due to a chemical (covalent bond formation) between the ligand and a its biological target.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

As used herein, the terms “treating” or “treatment” of a subject includes the administration of a drug to a subject with the purpose of preventing, curing, healing, alleviating, relieving, altering, remedying, ameliorating, improving, stabilizing or affecting a disease or disorder, or a symptom of a disease or disorder. The terms “treating” and “treatment” can also refer to reduction in severity and/or frequency of symptoms, elimination of symptoms and/or underlying cause, prevention of the occurrence of symptoms and/or their underlying cause, and improvement or remediation of damage. In particular, the term “treatment” includes the alleviation, in part or in whole, of the symptoms of coronavirus infection (e.g., sore throat, blocked and/or runny nose, cough and/or elevated temperature associated with a common cold). Such treatment may include eradication, or slowing of population growth, of a microbial agent associated with inflammation.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed. For example, the terms “prevent” or “suppress” can refer to a treatment that forestalls or slows the onset of a disease or condition or reduced the severity of the disease or condition. Thus, if a treatment can treat a disease in a subject having symptoms of the disease, it can also prevent or suppress that disease in a subject who has yet to suffer some or all of the symptoms. As used herein, the term “preventing” a disorder or unwanted physiological event in a subject refers specifically to the prevention of the occurrence of symptoms and/or their underlying cause, wherein the subject may or may not exhibit heightened susceptibility to the disorder or event. In particular embodiments, “prevention” includes reduction in risk of coronavirus infection in patients. However, it will be appreciated that such prevention may not be absolute, i.e., it may not prevent all such patients developing a coronavirus infection, or may only partially prevent an infection in a single individual. As such, the terms “prevention” and “prophylaxis” may be used interchangeably.

By the term “effective amount” of a therapeutic agent is meant a nontoxic but sufficient amount of a beneficial agent to provide the desired effect. The amount of beneficial agent that is “effective” will vary from subject to subject, depending on the age and general condition of the subject, the particular beneficial agent or agents, and the like. Thus, it is not always possible to specify an exact “effective amount”. However, an appropriate “effective” amount in any subject case may be determined by one of ordinary skill in the art using routine experimentation. Also, as used herein, and unless specifically stated otherwise, an “effective amount” of a beneficial can also refer to an amount covering both therapeutically effective amounts and prophylactically effective amounts.

An “effective amount” of a drug necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

As used herein, a “therapeutically effective amount” of a therapeutic agent refers to an amount that is effective to achieve a desired therapeutic result, and a “prophylactically effective amount” of a therapeutic agent refers to an amount that is effective to prevent an unwanted physiological condition. Therapeutically effective and prophylactically effective amounts of a given therapeutic agent will typically vary with respect to factors such as the type and severity of the disorder or disease being treated and the age, gender, and weight of the subject. The term “therapeutically effective amount” can also refer to an amount of a therapeutic agent, or a rate of delivery of a therapeutic agent (e.g., amount over time), effective to facilitate a desired therapeutic effect. The precise desired therapeutic effect will vary according to the condition to be treated, the tolerance of the subject, the drug and/or drug formulation to be administered (e.g., the potency of the therapeutic agent (drug), the concentration of drug in the formulation, and the like), and a variety of other factors that are appreciated by those of ordinary skill in the art.

As used herein, the term “pharmaceutically acceptable” component can refer to a component that is not biologically or otherwise undesirable, i.e., the component may be incorporated into a pharmaceutical formulation of the invention and administered to a subject as described herein without causing any significant undesirable biological effects or interacting in a deleterious manner with any of the other components of the formulation in which it is contained. When the term “pharmaceutically acceptable” is used to refer to an excipient, it is generally implied that the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

As used herein, “pharmaceutically acceptable salt” is a derivative of the disclosed compound in which the parent compound is modified by making inorganic and organic, non-toxic, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are typical, where practicable. Salts of the present compounds further include solvates of the compounds and of the compound salts.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH2)n-COOH where n is 0-4, and the like, or using a different acid that produces the same counterion. Lists of additional suitable salts may be found, e.g., in Remington's Pharmaceutical Sciences, 17th ed., Mack Publishing Company, Easton, Pa., p. 1418 (1985).

Also, as used herein, the term “pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

A “control” is an alternative subject or sample used in an experiment for comparison purposes. A control can be “positive” or “negative.”

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human. Thus, the subject can be a human or veterinary patient. The term “patient” refers to a subject under the treatment of a clinician, e.g., physician. Administration of the therapeutic agents can be carried out at dosages and for periods of time effective for treatment of a subject. In some embodiments, the subject is a human.

Reference will now be made in detail to specific aspects of the disclosed materials, compounds, compositions, articles, and methods, examples of which are illustrated in the accompanying Examples and Figures.

Pharmaceutical Compositions

Provided herein is a pharmaceutical composition including a paroxetine or a pharmaceutically acceptable salt or derivative thereof and one or more pharmaceutical acceptable carriers. In some embodiments, the compound is present in an effective amount to directly increase cAMP levels by at least 3 fold (e.g., at least 5 fold, at least 7 fold, at least 10 fold, at least 15 fold, at least 20 fold, at least 25 fold, at least 30 fold, at least 35 fold, or at least fold). In some embodiments, the compound is present in an effective amount to directly increase cAMP levels by 40 fold or less (e.g., 35 fold or less, 30 fold or less, 25 fold or less, 20 fold or less, 15 fold or less, 10 fold or less, 7 fold or less, or 5 fold or less). the compound is present in an effective amount to directly increase cAMP levels from any of the minimum values described above to any of the maximum values described above. For example, in some embodiments, the compound is present in an effective amount to directly increase cAMP levels from 3 fold to 40 fold (e.g., from 3 fold to 5 fold, from 3 fold to 7 fold, from 3 fold to 10 fold, from 3 fold to 15 fold, from 3 fold to 20 fold, from 3 fold to 25 fold, from 3 fold to 30 fold, from 3 fold to 35 fold, from 5 fold to 7 fold, from 5 fold to 10 fold, from 5 fold to 15 fold, from 5 fold to 20 fold, from 5 fold to 25 fold, from 5 fold to 30 fold, from 5 fold to 35 fold, from 5 fold to 40 fold, from 7 fold to 10 fold, from 7 fold to 15 fold, from 7 fold to 20 fold, from 7 fold to 25 fold, from 7 fold to 30 fold, from 7 fold to 35 fold, 7 fold to 40 fold, from 10 fold to 15 fold, from 10 fold to 20 fold, from 10 fold to 25 fold, from 10 fold to 30 fold, from 10 fold to 35 fold, 10 fold to 40 fold, from 10 fold to 20 fold, from 10 fold to 25 fold, from 10 fold to 30 fold, from 10 fold to 35 fold, 10 fold to 40 fold, from 15 fold to 20 fold, from 15 fold to 25 fold, from 15 fold to 30 fold, from 15 fold to 35 fold, 15 fold to 40 fold, from 15 fold to 20 fold, from 15 fold to 25 fold, from 15 fold to 30 fold, from 15 fold to 35 fold, 15 fold to 40 fold, from 20 fold to 25 fold, from 20 fold to 30 fold, from 20 fold to 35 fold, 20 fold to 40 fold, from 25 fold to 30 fold, from 25 fold to 35 fold, 25 fold to 40 fold, from 30 fold to 35 fold, 30 fold to 40 fold, or from 35 fold to 40 fold.

In some embodiments, the compound is present in an effective amount to increase PTH/PTH1R-mediated cAMP production by at least 2 fold, (e.g., at least 3 fold, at least 4 fold, at least 5 fold, at least 6 fold, at least 7 fold, at least 7 fold, or at least 8 fold). In some embodiments, the compound is present in an effective amount to increase PTH/PTH1R-mediated cAMP production by 8 fold or less (e.g., 7 fold or less, 6 fold or less, 5 fold or less, 4 fold or less, or 3 fold or less). The compound is present in an effective amount to increase PTH/PTH1R-mediated cAMP production from any of the minimum values described above to any of the maximum values described above. For example, in some embodiments, the compound is present in an effective amount to increase PTH/PTH1R-mediated cAMP production from 2 fold to 8 fold (e.g., from 2 fold to 6 fold, from 2 fold to 5 fold, from 2 fold to 6 fold, from 2 fold to 7 fold, from 3 fold to 4 fold, from 3 fold to 5 fold, from 3 fold to 6 fold, from 3 fold to 7 fold, from 4 fold to 5 fold, from 4 fold to 6 fold, from 4 fold to 7 fold, from 4 fold to 8 fold, from 5 fold to 6 fold, from 5 fold to 7 fold, from 5 fold to 8 fold, from 6 fold to 7 fold, from 6 fold to 8 fold, or from 7 fold to 8 fold). In some embodiments, the compound is present in an effective amount to increase PTH/PTH1R-mediated cAMP production by at least 2 fold.

In some embodiments, the PTH/PTH1R-mediated cAMP production is increased by inhibiting G-protein coupled receptor kinase 2 (GRK2). In some embodiments, the composition further includes parathyroid hormone (PTH).

The compositions described herein can be formulated for enteral, parenteral, topical, or pulmonary administration. The compounds can be combined with one or more pharmaceutically acceptable carriers and/or excipients that are considered safe and effective and may be administered to an individual without causing undesirable biological side effects or unwanted interactions. The carrier is all components present in the pharmaceutical formulation other than the active ingredient or ingredients.

Methods of Treating

Described herein are methods to treat inflammation resulting from an inflammatory disorder in a subject in need thereof, the method comprising administering to the subject a therapeutically effective amount of paroxetine or a pharmaceutically acceptable salt or derivative thereof or the pharmaceutical composition including paroxetine or a pharmaceutically acceptable salt or derivative thereof.

Described herein is also a method of inhibiting G-protein coupled receptor kinase 2 (GRK2) to increase PTH/PTH1R-mediated cAMP production including administering a therapeutically effective amount of a paroxetine or a pharmaceutically acceptable salt or derivative thereof or the pharmaceutical composition including paroxetine or a pharmaceutically acceptable salt or derivative thereof to a subject in need thereof.

In some embodiments, also provided are methods of increasing PTH/PTH1R-mediated cAMP production including administering a therapeutically effective amount of a paroxetine or a pharmaceutically acceptable salt or derivative thereof or the pharmaceutical composition to a subject in need thereof.

In some embodiments, the method is a method of treating osteoarthritis in a subject in need thereof, the method including administering a therapeutically effective amount of a paroxetine or a pharmaceutically acceptable salt or derivative thereof or the pharmaceutical composition including paroxetine or a pharmaceutically acceptable salt or derivative thereof to a subject in need thereof.

In some embodiments, the paroxetine or a pharmaceutically acceptable salt or derivative thereof or the pharmaceutical composition is present in a therapeutically effective amount to increase PTH/PTH1R-mediated cAMP production by at least 2 fold, (e.g., at least 3 fold, at least 4 fold, at least 5 fold, at least 6 fold, at least 7 fold, at least 7 fold, or at least 8 fold),In some embodiments, the paroxetine or a pharmaceutically acceptable salt or derivative thereof or the pharmaceutical composition is present in a therapeutically effective amount to increase PTH/PTH1R-mediated cAMP production by 8 fold or less (e.g., 7 fold or less, 6 fold or less, 5 fold or less, 4 fold or less, or 3 fold or less). The paroxetine or a pharmaceutically acceptable salt or derivative thereof or the pharmaceutical composition is present in a therapeutically effective amount to increase PTH/PTH1R-mediated cAMP production from any of the minimum values described above to any of the maximum values described above. For example, in some embodiments, the paroxetine or a pharmaceutically acceptable salt or derivative thereof or the pharmaceutical composition is present in a therapeutically effective amount to increase PTH/PTH1R-mediated cAMP production from 2 fold to 8 fold (e.g., from 2 fold to 6 fold, from 2 fold to 5 fold, from 2 fold to 6 fold, from 2 fold to 7 fold, from 3 fold to 4 fold, from 3 fold to fold, from 3 fold to 6 fold, from 3 fold to 7 fold, from 4 fold to 5 fold, from 4 fold to 6 fold, from 4 fold to 7 fold, from 4 fold to 8 fold, from 5 fold to 6 fold, from 5 fold to 7 fold, from 5 fold to 8 fold, from 6 fold to 7 fold, from 6 fold to 8 fold, or from 7 fold to 8 fold).

In some embodiments, the paroxetine or a pharmaceutically acceptable salt or derivative thereof or the pharmaceutical composition is present in a therapeutically effective amount to increase PTH/PTH1R-mediated cAMP production by at least 2 fold.

In some embodiments, a method for diminishing or ameliorating one or more symptoms caused by inflammation in a subject in need thereof is provided comprising administering a composition including paroxetine or a pharmaceutically acceptable salt or derivative thereof. In some embodiments, the method diminishes or ameliorates pain caused by osteoarthritis in a subject in need thereof. In some embodiments, the methods diminish or reduce cartilage degeneration. In some embodiments, the methods promote the regeneration of cartilage.

Described herein are also methods to treat pain caused by osteoarthritis in a subject in need thereof, the method comprising administering to the subject a therapeutically effective amount of paroxetine or a pharmaceutically acceptable salt or derivative thereof or the pharmaceutical composition including paroxetine or a pharmaceutically acceptable salt or derivative thereof.

In some embodiments, the inflammation comprises acute inflammation. In some embodiments, the acute inflammation may be in response to one or more of the following: a wound (such as a cut, bruise, or burn); exposure to a toxin or ionizing radiation; exposure to an allergen or antigen; and the presence of a foreign body (for example, a splinter) in a subject.

In some embodiments, the inflammation comprises chronic inflammation. In some embodiments, the chronic inflammation may be associated with a persistent form of acute inflammation, as described above, or may be associated with an inflammatory disorder.

The present methods may be used to treat or prevent inflammation in any part of the body, including but not limited to inflammation of: the central nervous system (such as encephalitis, myelitis, or meningitis); the peripheral nervous system (such as neuritis); the eye (such as dacryoadenitis, scleritis, episcleritis, or keratitis); the ear (such as otitis); the heart (such as endocarditis, myocarditis, or pericarditis); the vascular system (such as arteritis, phlebitis, or capillaritis); the respiratory system (such as sinusitis, rhinitis, pharyngitis, epiglottitis, laryngitis, tracheitis, bronchitis, pneumonitis, or pleurisy); the digestive system (such as stomatitis, gingivitis, glossitis, tonsillitis, sialadenitis, parotitis, cheilitis, pulpitis, gnathitis, oesophagitis, gastritis, gastroenteritis, enteritis, colitis, pancolitis, appendicitis, cryptitis, hepatitis, cholecystitis, or pancreatitis); the integumentary system (such as dermatitis or mastitis); the musculoskeletal system (such as arthritis, myositis, synovitis, tenosynovitis, or bursitis); the urinary system (such as nephritis, ureteritis, cystitis, or urethritis); the female reproductive system (such as oophoritis, salpingitis, endometritis, myometritis, parametritis, cervicitis, vaginitis, or vulvitis); the male reproductive system (such as orchitis, epididymitis, prostatitis, vasculitis, balanitis, or posthitis); the endocrine system (such as insulitis, hypophysitis, thyroiditis, parathyroiditis, or adrenalitis); or the lymphatic system (such a lymphangitis or lymphadenitis).

The present methods may also be used to treat or prevent inflammation resulting from an inflammatory disorder. In some embodiments, the methods described herein may be used to treat arthritis, including but not limited to rheumatoid arthritis, spondyloarthopathies, gouty arthritis, systemic lupus erythematosus, psoriatic arthritis, osteoarthritis, and juvenile arthritis. In some embodiments, the methods described herein may be used to treat asthma, bronchitis, menstrual cramps, tendinitis, bursitis, and skin related conditions such as psoriasis, eczema, burns and dermatitis. In some embodiments, the methods described herein may be used to treat gastrointestinal conditions such as inflammatory bowel disease, Crohn's disease, gastritis, irritable bowel syndrome, and ulcerative colitis. In some embodiments, the methods described herein may be used to treat inflammation present in a disorder including, but not limited to, vascular disease, migraine headaches, perarteritis nodosa, thyroiditis, aplastic anemia, Hodgkin's disease, scleroderma, rheumatic fever, type I diabetes, myasthenia gravis, sarcoidosis, nephrotic syndrome, Behcet's syndrome, polymyositis, hypersensitivity, conjunctivitis, gingivitis, swelling occurring after an injury, myocardial ischemia, and the like.

In some embodiments, the methods described herein may be used to treat or prevent inflammation associated with a disorder including, but not limited to, acne vulgaris, asthma, an autoimmune disease, an autoinflammatory disease, celiac disease, chronic prostatitis, colitis, diverticulitis, glomerulonephritis, hidradenitis suppurativa, hypersensitivities, inflammatory bowel disease, interstitial cystitis, lichen planus, mast cell activation syndrome, otitis, pelvic inflammatory disease, reperfusion injury, rheumatic fever, osteoarthritis, rheumatoid arthritis, rhinitis, sarcoidosis, transplant rejection, or vasculitis. In some embodiments, the methods described herein may be used to treat or prevent inflammation associated with atherosclerosis, cancer, or ischemic heart disease.

In some embodiments, the methods described herein may be used to treat a systemic inflammatory disorder or ameliorate or diminish one or more inflammatory symptoms of a system inflammatory disorder including, but not limited to, non-alcoholic fatty liver disease, non-alcoholic steatohepatitis, inflammatory bowel disease, Crohn's disease, ulcerative colitis, psoriasis, irritable bowel syndrome, arthritis, ankylosing spondylitis, osteoporosis, rheumatoid arthritis, psoriatic arthritis, osteoarthritis, chronic obstructive pulmonary disease, atherosclerosis, pulmonary arterial hypertension, pyridoxine-dependent epilepsy, atopic dermatitis, rosacea, multiple sclerosis, systemic lupus erythematosus, lupus nephritis, sepsis, eosinophilic esophagitis, chronic kidney disease, fibrotic renal disease, chronic eosinophilic pneumonia, extrinsic allergic alveolitis, pre-eclampsia, endometriosis, polycystic ovary syndrome, or cyclophosphamide-induced hemorrhagic cystitis.

In some embodiments, the methods described herein may be used to treat inflammation resulting from a disorder selected from light chain deposition disease, IgA nephropathy, end-stage renal disease, gout, pseudogout, diabetic nephropathy, diabetic neuropathy, traumatic brain injury, noise-induced hearing loss, Alzheimer's disease, Parkinson's disease, Huntington disease, amyotrophic lateral sclerosis, primary biliary cirrhosis, primary sclerosing cholangitis, uterine leiomyoma, sarcoidosis, or chronic kidney disease.

### Combination Therapy

In some embodiments, the compositions as used in the methods described herein can further comprise or be administered in combination with other therapies. The composition described herein can be administered simultaneously, sequentially, or at distinct time points as part of the same therapeutic regimen.

In some embodiments, the composition as used in the methods described herein can further comprise or be administered in combination with acetaminophen (paracetamol).

In some embodiments, the composition as used in the methods described herein can further comprise or be administered in combination with a non-steroidal anti-inflammatory drug, including but not limited to: aspirin, diflunisal, salicylic acid and its salts, salsalate, ibuprofen, fenoprofen, flurbiprofen, dexibuprofen, ketoprofen, oxaprozin, naproxen, dexketoprofen, loxoprofen, indomethacin, etodolac, aceclofenac, tolmetin, ketorolac, nabumetone, sulindac, diclofenac, piroxicam, tenoxicam, lornoxicam, phenylbutazone, meloxicam, droxicam, isoxicam, mefenamic acid, meclofenamic acid, flufenamic acid, tolfenamic acid, celecoxib, valdecoxib, lumiracoxib, firocoxib, rofecoxib, parecoxib, etoricoxib, nimesulide, clonixin, licofelone, and harpagide.

In some embodiments, the composition as used in the methods described herein can further comprise or be administered in combination with an opioid, including but not limited to: opium alkaloids and derivatives such as codeine, thebaine, morphine, oripavine, diacetylmorphine, di acetyl dihydromorphine, methyldesorphine, nicomorphine, acetylpropionylmorphine, dibenzoylmorphine, dipropanoylmorphine, desomorphine, dihydrocodeine, ethylmorphine, heterocodeine, buprenorphine, hydrocodone, oxycodone, etorphine, hydromorphone, oxymorphone, fentanyl, sufentanil, ohmefentanyl, alphamethylfentanyl, remifentanil, alfentanil, carfentanyl, pethidine, allylprodine, promedol ketobemidone, prodine, desmethylprodine, phenethylphenylacetoxypiperidine, propoxyphene, methadone, loperamide, dextropropoxyphene, dipianone, dextromoramide, levomethadyl acetate, bezitramide, difenoxin, piritramide, diphenoxylate, dezocine, pentazocine, phenazocine, buprenorphine, dihydroetorphine, etorphine, butorphanol, levorphanol, racemethorphan, nalbuphine, levomethorphan, lefetamine, tilidine, buccinazine, menthol, tramadol, 7-hydroxymitragynine, meptazinol, tapentadol, mitragynine, or eluxadoline; or opioid antagonists such as nalmefene, methylnaltrexone, naloxegol, naloxone, or naltrexone.

In some embodiments, the composition as used in the methods described herein can further comprise or be administered in combination with an antidepressant, including but not limited to: fluoxetine, duloxetine, venlafaxine, milnacipran, amitriptyline, nortriptypine, desipramine, or bupropion.

In some embodiments, the composition as used in the methods described herein can further comprise or be administered in combination with an anticonvulsant, including but not limited to: pregabalin, gabapentin, carbamazepine, or oxcarbazepine.

In some embodiments, the composition as used in the methods described herein can further comprise or be administered in combination with a topical anesthetic, including but not limited to: benzocaine, butamben, dibucaine, lidocaine, oxybuprocaine, pramoxine, proparacaine, proxymetacaine, and tetracaine. In some embodiments, the composition as used in the methods described herein can further comprise or be administered in combination with capsaicin.

In some embodiments, the composition as used in the methods described herein can further comprise or be administered in combination with caffeine.

In some embodiments, the composition as used in the methods described herein can further comprise or be administered in combination with an N-methyl-D-aspartate receptor antagonist including, but not limited to: memantine, ketamine, or dextromethorphan.

Methods of Administration

The compositions as used in the methods described herein can be administered by any suitable method and technique presently or prospectively known to those skilled in the art. For example, the active components described herein can be formulated in a physiologically- or pharmaceutically-acceptable form and administered by any suitable route known in the art including, for example, oral and parenteral routes of administering. As used herein, the term “parenteral” includes subcutaneous, intradermal, intravenous, intramuscular, intraperitoneal, and intrasternal administration, such as by injection. Administration of the active components of their compositions can be a single administration, or at continuous and distinct intervals as can be readily determined by a person skilled in the art.

Compositions, as described herein, comprising an active compound and an excipient of some sort may be useful in a variety of medical and non-medical applications. For example, pharmaceutical compositions comprising an active compound and an excipient may be useful for the treatment of osteoarthritis.

“Excipients” include any and all solvents, diluents or other liquid vehicles, dispersion or suspension aids, surface active agents, isotonic agents, thickening or emulsifying agents, preservatives, solid binders, lubricants and the like, as suited to the particular dosage form desired. General considerations in formulation and/or manufacture can be found, for example, in Remington's Pharmaceutical Sciences, Sixteenth Edition, E. W. Martin (Mack Publishing Co., Easton, Pa., 1980), and Remington: The Science and Practice of Pharmacy, 21st Edition (Lippincott Williams & Wilkins, 2005).

Exemplary excipients include, but are not limited to, any non-toxic, inert solid, semisolid or liquid filler, diluent, encapsulating material or formulation auxiliary of any type. Some examples of materials which can serve as excipients include, but are not limited to, sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. As would be appreciated by one of skill in this art, the excipients may be chosen based on what the composition is useful for. For example, with a pharmaceutical composition or cosmetic composition, the choice of the excipient will depend on the route of administration, the agent being delivered, time course of delivery of the agent, etc., and can be administered to humans and/or to animals, orally, rectally, parenterally, intracisternally, intravaginally, intranasally, intraperitoneally, topically (as by powders, creams, ointments, or drops), buccally, or as an oral or nasal spray. In some embodiments, the active compounds disclosed herein are administered topically.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and combinations thereof.

Exemplary granulating and/or dispersing agents include potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and combinations thereof.

Exemplary surface active agents and/or emulsifiers include natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. Exemplary binding agents include starch (e.g. cornstarch and starch paste), gelatin, sugars (e.g. sucrose, glucose, dextrose, dextrin, molasses, lactose, lactitol, mannitol, etc.), natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, etc., and/or combinations thereof.

Exemplary preservatives include antioxidants, chelating agents, antimicrobial preservatives, antifungal preservatives, alcohol preservatives, acidic preservatives, and other preservatives.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid. Other preservatives include tocopherol, tocopherol acetate, deteroxime mesylate, cetrimide, butylated hydroxyanisol (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl. In certain embodiments, the preservative is an anti-oxidant. In other embodiments, the preservative is a chelating agent.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and combinations thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary natural oils include almond, apricot kernel, avocado, babassu, bergamot, black current seed, borage, cade, chamomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof.

Additionally, the composition may further comprise a polymer. Exemplary polymers contemplated herein include, but are not limited to, cellulosic polymers and copolymers, for example, cellulose ethers such as methylcellulose (MC), hydroxyethylcellulose (HEC), hydroxypropyl cellulose (HPC), hydroxypropyl methyl cellulose (HPMC), methylhydroxyethylcellulose (MHEC), methylhydroxypropylcellulose (MHPC), carboxymethyl cellulose (CMC) and its various salts, including, e.g., the sodium salt, hydroxy ethyl carboxymethylcellulose (HECMC) and its various salts, carboxymethylhydroxyethylcellulose (CMHEC) and its various salts, other polysaccharides and polysaccharide derivatives such as starch, dextran, dextran derivatives, chitosan, and alginic acid and its various salts, carageenan, various gums, including xanthan gum, guar gum, gum arabic, gum karaya, gum ghatti, konjac and gum tragacanth, glycosaminoglycans and proteoglycans such as hyaluronic acid and its salts, proteins such as gelatin, collagen, albumin, and fibrin, other polymers, for example, polyhydroxyacids such as polylactide, polyglycolide, polyl(lactide-co-glycolide) and poly(.epsilon.-caprolactone-co-glycolide)-, carboxyvinyl polymers and their salts (e.g., carbomer), polyvinylpyrrolidone (PVP), polyacrylic acid and its salts, polyacrylamide, polyacrylic acid/acrylamide copolymer, polyalkylene oxides such as polyethylene oxide, polypropylene oxide, poly(ethylene oxide-propylene oxide), and a Pluronic polymer, polyoxy ethylene (polyethylene glycol), polyanhydrides, polyvinylalchol, polyethyleneamine and polypyrridine, polyethylene glycol (PEG) polymers, such as PEGylated lipids (e.g., PEG-stearate, 1,2-Distearoyl-sn-glycero-3-Phosphoethanolamine-N-[Methoxy(Polyethylene glycol)-1000], 1,2-Distearoyl-sn-glycero-3-Phosphoethanolamine-N-[Methoxy(Polyethylene glycol)-2000], and 1,2-Distearoyl-sn-glycero-3-Phosphoethanolamine-N-[Methoxy(Polyethylene glycol)-5000]), copolymers and salts thereof.

Additionally, the composition may further comprise an emulsifying agent. Exemplary emulsifying agents include, but are not limited to, a polyethylene glycol (PEG), a polypropylene glycol, a polyvinyl alcohol, a poly-N-vinyl pyrrolidone and copolymers thereof, poloxamer nonionic surfactants, neutral water-soluble polysaccharides (e.g., dextran, Ficoll, celluloses), non-cationic poly(meth)acrylates, non-cationic polyacrylates, such as poly (meth) acrylic acid, and esters amide and hydroxy alkyl amides thereof, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. In certain embodiments, the emulsifying agent is cholesterol.

Liquid compositions include emulsions, microemulsions, solutions, suspensions, syrups, and elixirs. In addition to the active compound, the liquid composition may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable compositions, for example, injectable aqueous or oleaginous suspensions may be formulated according to the known art using suitable dispersing or wetting agents and suspending agents. The sterile injectable preparation may also be an injectable solution, suspension, or emulsion in a nontoxic parenterally acceptable diluent or solvent, for example, as a solution in 1,3-butanediol. Among the acceptable vehicles and solvents for pharmaceutical or cosmetic compositions that may be employed are water, Ringer's solution, U.S.P. and isotonic sodium chloride solution. In addition, sterile, fixed oils are conventionally employed as a solvent or suspending medium. Any bland fixed oil can be employed including synthetic mono- or diglycerides. In addition, fatty acids such as oleic acid are used in the preparation of injectables. In certain embodiments, the particles are suspended in a carrier fluid comprising 1% (w/v) sodium carboxymethyl cellulose and 0.1% (v/v) Tween 80. The injectable composition can be sterilized, for example, by filtration through a bacteria-retaining filter, or by incorporating sterilizing agents in the form of sterile solid compositions which can be dissolved or dispersed in sterile water or other sterile injectable medium prior to use.

Compositions for rectal or vaginal administration may be in the form of suppositories which can be prepared by mixing the particles with suitable non-irritating excipients or carriers such as cocoa butter, polyethylene glycol, or a suppository wax which are solid at ambient temperature but liquid at body temperature and therefore melt in the rectum or vaginal cavity and release the particles.

Solid compositions include capsules, tablets, pills, powders, and granules. In such solid compositions, the particles are mixed with at least one excipient and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets, and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

Tablets, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes. Solid compositions of a similar type may also be employed as fillers in soft and hard-filled gelatin capsules using such excipients as lactose or milk sugar as well as high molecular weight polyethylene glycols and the like.

Compositions for topical or transdermal administration include ointments, pastes, creams, lotions, gels, powders, solutions, sprays, inhalants, or patches. The active compound is admixed with an excipient and any needed preservatives or buffers as may be required.

The ointments, pastes, creams, and gels may contain, in addition to the active compound, excipients such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to the active compound, excipients such as lactose, talc, silicic acid, aluminum hydroxide, calcium silicates, and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the nanoparticles in a proper medium. Absorption enhancers can also be used to increase the flux of the compound across the skin. The rate can be controlled by either providing a rate controlling membrane or by dispersing the particles in a polymer matrix or gel.

The compounds can be incorporated microparticles, nanoparticles, or combinations thereof that provide controlled release of the compounds and/or additional active agents. For example, the compounds can be incorporated into polymeric microparticles, which provide controlled release of the drug(s). Release of the drug(s) is controlled by diffusion of the drug(s) out of the microparticles and/or degradation of the polymeric particles by hydrolysis and/or enzymatic degradation. Suitable polymers include ethylcellulose and other natural or synthetic cellulose derivatives.

Polymers, which are slowly soluble and form a gel in an aqueous environment, such as hydroxypropyl methylcellulose or polyethylene oxide, may also be suitable as materials for drug containing microparticles. Other polymers include, but are not limited to, polyanhydrides, poly(ester anhydrides), polyhydroxy acids, such as polylactide (PLA), polyglycolide (PGA), poly(lactide-co-glycolide) (PLGA), poly-3-hydroxybutyrate (PHB) and copolymers thereof, poly-4-hydroxybutyrate (P4HB) and copolymers thereof, polycaprolactone and copolymers thereof, and combinations thereof.

Alternatively, the compound can be incorporated into microparticles prepared from materials which are insoluble in aqueous solution or slowly soluble in aqueous solution, but are capable of degrading within the GI tract by means including enzymatic degradation, surfactant action of bile acids, and/or mechanical erosion. As used herein, the term “slowly soluble in water” refers to materials that are not dissolved in water within a period of 30 minutes. Preferred examples include fats, fatty substances, waxes, wax-like substances and mixtures thereof. Suitable fats and fatty substances include fatty alcohols (such as lauryl, myristyl stearyl, cetyl or cetostearyl alcohol), fatty acids and derivatives, including but not limited to fatty acid esters, fatty acid glycerides (mono-, di- and tri-glycerides), and hydrogenated fats. Specific examples include, but are not limited to hydrogenated vegetable oil, hydrogenated cottonseed oil, hydrogenated castor oil, hydrogenated oils available under the trade name Sterotex®, stearic acid, cocoa butter, and stearyl alcohol. Suitable waxes and wax-like materials include natural or synthetic waxes, hydrocarbons, and normal waxes. Specific examples of waxes include beeswax, glycowax, castor wax, carnauba wax, paraffins and candelilla wax. As used herein, a wax-like material is defined as any material, which is normally solid at room temperature and has a melting point of from about 30 to 300° C.

In some cases, it may be desirable to alter the rate of water penetration into the microparticles. To this end, rate-controlling (wicking) agents may be formulated along with the fats or waxes listed above. Examples of rate-controlling materials include certain starch derivatives (e.g., waxy maltodextrin and drum dried corn starch), cellulose derivatives (e.g., hydroxypropylmethyl-cellulose, hydroxypropylcellulose, methylcellulose, and carboxymethyl-cellulose), alginic acid, lactose and talc. Additionally, a pharmaceutically acceptable surfactant (for example, lecithin) may be added to facilitate the degradation of such microparticles.

Proteins, which are water insoluble, such as zein, can also be used as materials for the formation of drug containing microparticles. Additionally, proteins, polysaccharides and combinations thereof, which are water-soluble, can be formulated with drug into microparticles and subsequently cross-linked to form an insoluble network. For example, cyclodextrins can be complexed with individual drug molecules and subsequently cross-linked.

Encapsulation or incorporation of drug into carrier materials to produce drug-containing microparticles can be achieved through known pharmaceutical formulation techniques. In the case of formulation in fats, waxes or wax-like materials, the carrier material is typically heated above its melting temperature and the drug is added to form a mixture comprising drug particles suspended in the carrier material, drug dissolved in the carrier material, or a mixture thereof. Microparticles can be subsequently formulated through several methods including, but not limited to, the processes of congealing, extrusion, spray chilling or aqueous dispersion. In a preferred process, wax is heated above its melting temperature, drug is added, and the molten wax-drug mixture is congealed under constant stirring as the mixture cools. Alternatively, the molten wax-drug mixture can be extruded and spheronized to form pellets or beads. These processes are known in the art.

For some carrier materials it may be desirable to use a solvent evaporation technique to produce drug-containing microparticles. In this case drug and carrier material are co-dissolved in a mutual solvent and microparticles can subsequently be produced by several techniques including, but not limited to, forming an emulsion in water or other appropriate media, spray drying or by evaporating off the solvent from the bulk solution and milling the resulting material.

In some embodiments, drug(s) in a particulate form is homogeneously dispersed in a water-insoluble or slowly water soluble material. To minimize the size of the drug particles within the composition, the drug powder itself may be milled to generate fine particles prior to formulation. The process of jet milling, known in the pharmaceutical art, can be used for this purpose. In some embodiments, drug in a particulate form is homogeneously dispersed in a wax or wax like substance by heating the wax or wax like substance above its melting point and adding the drug particles while stirring the mixture. In this case a pharmaceutically acceptable surfactant may be added to the mixture to facilitate the dispersion of the drug particles.

The particles can also be coated with one or more modified release coatings. Solid esters of fatty acids, which are hydrolyzed by lipases, can be spray coated onto microparticles or drug particles. Zein is an example of a naturally water-insoluble protein. It can be coated onto drug containing microparticles or drug particles by spray coating or by wet granulation techniques. In addition to naturally water-insoluble materials, some substrates of digestive enzymes can be treated with cross-linking procedures, resulting in the formation of non-soluble networks. Many methods of cross-linking proteins, initiated by both chemical and physical means, have been reported. One of the most common methods to obtain cross-linking is the use of chemical cross-linking agents. Examples of chemical cross-linking agents include aldehydes (gluteraldehyde and formaldehyde), epoxy compounds, carbodiimides, and genipin. In addition to these cross-linking agents, oxidized and native sugars have been used to cross-link gelatin. Cross-linking can also be accomplished using enzymatic means; for example, transglutaminase has been approved as a GRAS substance for cross-linking seafood products. Finally, cross-linking can be initiated by physical means such as thermal treatment, UV irradiation and gamma irradiation.

To produce a coating layer of cross-linked protein surrounding drug containing microparticles or drug particles, a water-soluble protein can be spray coated onto the microparticles and subsequently cross-linked by the one of the methods described above. Alternatively, drug-containing microparticles can be microencapsulated within protein by coacervation-phase separation (for example, by the addition of salts) and subsequently cross-linked. Some suitable proteins for this purpose include gelatin, albumin, casein, and gluten.

Polysaccharides can also be cross-linked to form a water-insoluble network. For many polysaccharides, this can be accomplished by reaction with calcium salts or multivalent cations, which cross-link the main polymer chains. Pectin, alginate, dextran, amylose and guar gum are subject to cross-linking in the presence of multivalent cations. Complexes between oppositely charged polysaccharides can also be formed; pectin and chitosan, for example, can be complexed via electrostatic interactions.

In certain embodiments, it may be desirable to provide continuous delivery of one or more compounds to a patient in need thereof. For intravenous or intraarterial routes, this can be accomplished using drip systems, such as by intravenous administration. For topical applications, repeated application can be done or a patch can be used to provide continuous administration of the compounds over an extended period of time.

The compounds described herein can be incorporated into injectable/implantable solid or semi-solid implants, such as polymeric implants. In one embodiment, the compounds are incorporated into a polymer that is a liquid or paste at room temperature, but upon contact with aqueous medium, such as physiological fluids, exhibits an increase in viscosity to form a semi-solid or solid material. Exemplary polymers include, but are not limited to, hydroxyalkanoic acid polyesters derived from the copolymerization of at least one unsaturated hydroxy fatty acid copolymerized with hydroxyalkanoic acids. The polymer can be melted, mixed with the active substance and cast or injection molded into a device. Such melt fabrication require polymers having a melting point that is below the temperature at which the substance to be delivered and polymer degrade or become reactive. The device can also be prepared by solvent casting where the polymer is dissolved in a solvent and the drug dissolved or dispersed in the polymer solution and the solvent is then evaporated. Solvent processes require that the polymer be soluble in organic solvents. Another method is compression molding of a mixed powder of the polymer and the drug or polymer particles loaded with the active agent.

Alternatively, the compounds can be incorporated into a polymer matrix and molded, compressed, or extruded into a device that is a solid at room temperature. For example, the compounds can be incorporated into a biodegradable polymer, such as polyanhydrides, polyhydroalkanoic acids (PHAs), PLA, PGA, PLGA, polycaprolactone, polyesters, polyamides, polyorthoesters, polyphosphazenes, proteins and polysaccharides such as collagen, hyaluronic acid, albumin and gelatin, and combinations thereof and compressed into solid device, such as disks, wafers, or extruded into a device, such as rods.

The release of the compounds from the implant can be varied by selection of the polymer, the molecular weight of the polymer, and/or modification of the polymer to increase degradation, such as the formation of pores and/or incorporation of hydrolyzable linkages. Methods for modifying the properties of biodegradable polymers to vary the release profile of the compounds from the implant are well known in the art.

In some embodiments, the compounds or pharmaceutical compositions can be administered locally. In some embodiments, the compounds are incorporated in a delivery system such as gels, nanoparticles, microparticles, or implants such as (e.g., rods, discs, wafers, orthopedic implants) for sustained release. In some embodiments, the compounds can be administered using a local delivery implantable system comprising the compounds incorporated within a gel, nanoparticles, microparticles, or an implant. In some embodiments, the pharmaceutical compositions comprise a delivery system such as gels, nanoparticles, microparticles, or implants such as (e.g., rods, discs, wafers, orthopedic implants) for sustained release of paroxetine or a pharmaceutically acceptable salt or derivative thereof.

The active ingredient may be administered in such amounts, time, and route deemed necessary in order to achieve the desired result. The exact amount of the active ingredient will vary from subject to subject, depending on the species, age, and general condition of the subject, the severity of the infection, the particular active ingredient, its mode of administration, its mode of activity, and the like. The active ingredient, whether the active compound itself, or the active compound in combination with an agent, is preferably formulated in dosage unit form for ease of administration and uniformity of dosage. It will be understood, however, that the total daily usage of the active ingredient will be decided by the attending physician within the scope of sound medical judgment. The specific therapeutically effective dose level for any particular subject will depend upon a variety of factors including the disorder being treated and the severity of the disorder; the activity of the active ingredient employed; the specific composition employed; the age, body weight, general health, sex and diet of the patient; the time of administration, route of administration, and rate of excretion of the specific active ingredient employed; the duration of the treatment; drugs used in combination or coincidental with the specific active ingredient employed; and like factors well known in the medical arts.

The active ingredient may be administered by any route. In some embodiments, the active ingredient is administered via a variety of routes, including oral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, enteral, sublingual; by intratracheal instillation, bronchial instillation, and/or inhalation; and/or as an oral spray, nasal spray, and/or aerosol. In general, the most appropriate route of administration will depend upon a variety of factors including the nature of the active ingredient (e.g., its stability in the environment of the gastrointestinal tract), the condition of the subject (e.g., whether the subject is able to tolerate oral administration), etc.

The exact amount of an active ingredient required to achieve a therapeutically or prophylactically effective amount will vary from subject to subject, depending on species, age, and general condition of a subject, severity of the side effects or disorder, identity of the particular compound(s), mode of administration, and the like. The amount to be administered to, for example, a child or an adolescent can be determined by a medical practitioner or person skilled in the art and can be lower or the same as that administered to an adult.

Useful dosages of the active agents and pharmaceutical compositions disclosed herein can be determined by comparing their in vitro activity, and in vivo activity in animal models. Methods for the extrapolation of effective dosages in mice, and other animals, to humans are known to the art.

The dosage ranges for the administration of the compositions are those large enough to produce the desired effect in which the symptoms or disorder are affected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counterindications. Dosage can vary, and can be administered in one or more dose administrations daily, for one or several days.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### EXAMPLES

### Example 1: GRK2 Inhibition Promotes PTH-PTH1R Chondroprotection in Articular Chondrocytes Following DMM

Introduction: Osteoarthritis (OA) is a complicated degenerative disease of the joint with chondrocyte hypertrophy (CH) being the principle cellular process leading to cartilage degeneration. While there is no treatment that can stop cartilage degeneration in OA, parathyroid hormone (PTH) has demonstrated chondroprotective and chondroregenerative effects in mice. PTH works through parathyroid hormone receptor type-1 (PTH1R), a GPCR expressed in growth plate and articular chondrocytes. In growth plate chondrocytes, PTH1R-Gas signaling maintains chondrocyte homeostasis and prevents their hypertrophy. We have previously shown that GPCR GRK2 signaling is elevated in other diseases leading to GPCR desensitization and the loss of Ga signaling, and that inhibition of GRK2 attenuates disease progression. However, the role of GPCR GRK2 signaling in OA remains unknown. We hypothesize that elevated chondrocyte GRK2 signaling in OA leads to PTH1R desensitization and subsequent loss of PTH mediated chondroprotection, and that GRK2 inhibition can rescue PTH1R by preventing its desensitization, thus attenuate chondrocyte hypertrophy and promote the chondroprotective effects of PTH. This study aims to, (1) determine changes in PTH1R-Gas signaling in OA and (2) determine the role of GRK2 inhibition in PTH1R resensitization and PTH-mediated chondroprotection in OA.

Methods: Destabilization of the medial meniscus (DMM) surgery was used to simulate clinical post-traumatic OA. First, we determined the role of GRK2 inhibition in PTH1R Gas-cAMP signaling: 12 week old male mice underwent sham or DMM surgery and were treated with either vehicle or the GRK2 inhibitor “paroxetine” (ip, 5 mg/kg/day), treatment was initiated at the time of surgery and continued daily until 8 weeks. Knees were harvested and articular cartilage was obtained for ex vivo analysis of Gas activity. In ex vivo culture, cartilage explants were treated with PTH as an agonist to stimulate PTH1R-Ga-cAMP signaling. Thirty minutes following PTH stimulation, cartilage tissue was homogenized and cAMP levels were measured. Then, we determined the role of GRK2 inhibition in recovering or even potentiating PTH1R signaling in OA cartilage: 12 week old male mice underwent sham or DMM surgery, and received treatment with vehicle, PTH (sc, 40 μg/kg/day) alone, the GRK2 inhibitor “paroxetine3” (ip, 5 mg/kg/day) alone, or their combination. Treatment was initiated at week 8 following DMM surgery, where OA is in progress, and continued until week 12 where mice were sacrificed. Knees were harvested, paraffin embedded, sectioned and stained with safranin-o and fast green for histological evaluation, OARSI scoring, and histomorphometric analysis. Results are expressed as mean values+s.e.m. with an N=5-8 mice per experimental group. Statistical analyses were performed using one ANVOA with P<0.05 representing statistical significance.

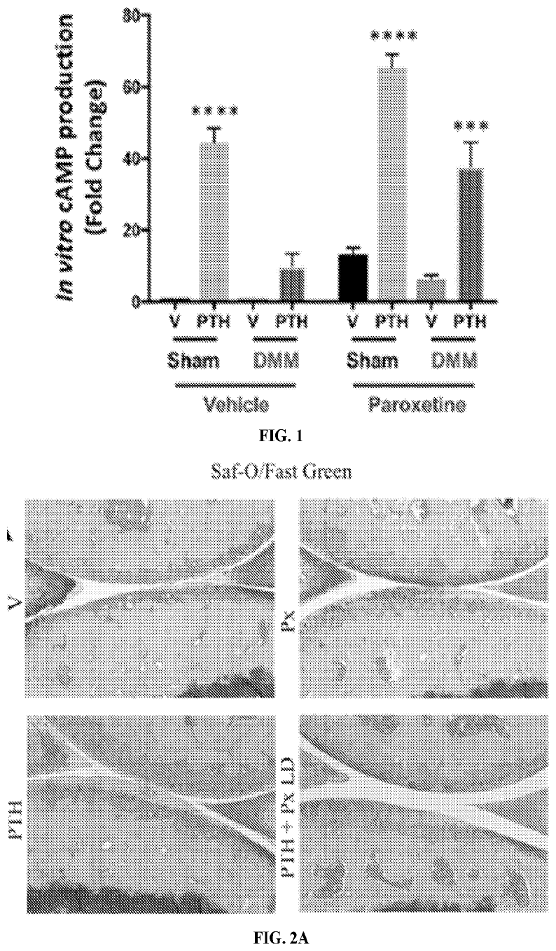

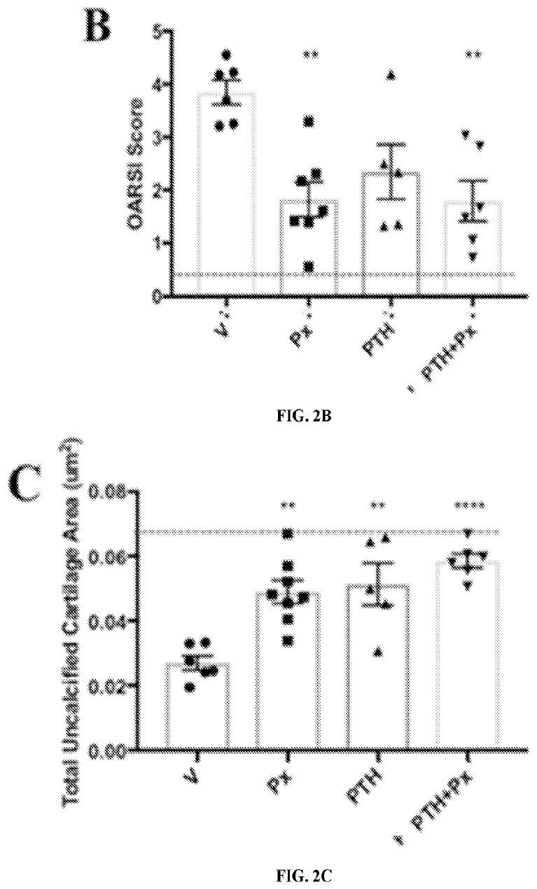

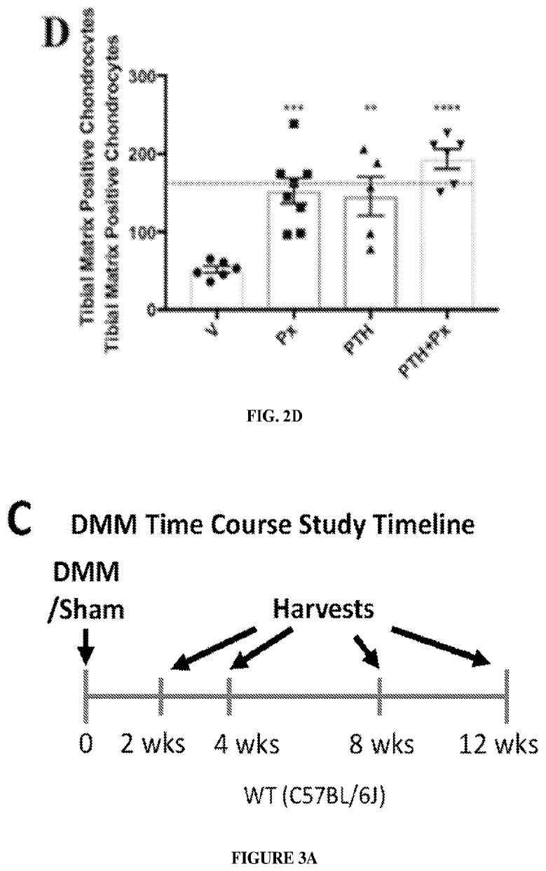

Results: In sham mouse cartilage, PTH/PTH1R signaling yielded high levels of cAMP production, while in DMM mouse cartilage, PTH/PTH1R-mediated cAMP production was very low, suggesting desensitization of PTH1R and loss of its Gas signaling in DMM mice. Interestingly, treatment with the GRK2 inhibitor paroxetine potentiated PTH/PTH1R-mediated cAMP production in sham mice and recovered it in DMM mice (FIG. 1). Further, in DMM mice, treatment with either PTH or paroxetine alone increased uncalcified cartilage area and chondrocyte number, suggesting a chondroprotective effect. We found a particular increase in the number of matrix-producing (anabolic chondrocytes), suggesting a chondroregenerative effect. Importantly, combining PTH treatment with GRK2 inhibition (by paroxetine) demonstrated an additive therapeutic effect, suggesting a potentiation of PTH/PTH1R chondroprotective and chondroregenerative signaling by paroxetine (FIG. 2).

Discussion: In DMM mice, chondrocyte PTH1R is desensitized, as evidenced by the great loss of Gas-cAMP production following PTH stimulation. This desensitization is primarily mediated through GRK2, since GRK2 inhibition lead to the recovery of cAMP production in DMM mice and potentiated it in sham mice. In progressing OA, treatment with either PTH or paroxetine is therapeutically effective and prevented cartilage area loss, chondrocyte hypertrophy and loss. This suggests a chondroprotective effect, and promoted chondrocyte anabolic phenotype suggesting a chondroregenerative effect. However, the combination of both drugs had a synergistic chondroprotective and chondroregerative effect. This synergy is probably due to PTH1R resensitization by paroxetine, which increases the efficacy of PTH treatment by increasing PTH1R availability for ligand-receptor interaction. In conclusion, elevated GRK2 signaling plays an important regulatory role in the loss of PTH mediated chondroprotection through PTH1R-Gas signaling in articular cartilage following injury, and GRK2 inhibition recovers PTH1R signaling and potentiates PTH therapeutic effect in OA.

Significance: Further understanding of chondrocyte regulation can aid in the development of therapeutic options for osteoarthritis patients. Current treatments for OA primarily focus on palliative pain management therapies with little potential to prevent further articular cartilage degeneration. PTH is widely used in treatment of other musculoskeletal diseases, with a recently identified therapeutic effect in OA. Here, we (1) identify a major mechanism for the chondroprotective effect of PTH in OA, and (2) identify a therapeutic combination that potentiates the chondroprotective and chondroregenerative effects of PTH in OA, which may enable the use of lower clinical doses of PTH to avoid some of its side effects.

### Example 2: Paroxetine-Mediated GRK2 Inhibition, a Novel Disease Modifying Treatment for Osteoarthritis

Abstract

Osteoarthritis (OA) is a debilitating joint disease characterized by progressive cartilage degeneration, with no available disease-modifying therapy. OA is driven by pathological chondrocyte hypertrophy (CH), whose cellular regulators are unknown. We have recently reported the therapeutic efficacy of G-protein coupled receptor kinase 2 (GRK2) inhibition in other diseases by recovering protective G-protein coupled receptor (GPCR) signaling. However, the role of GPCR-GRK2 pathway in OA is unknown. Thus, in a surgical OA mouse model, we performed genetic GRK2 deletion in chondrocytes, or pharmacological inhibition with the repurposed FDA approved antidepressant paroxetine. Both GRK2 deletion and inhibition prevented CH, abated OA progression, and promoted cartilage regeneration. Supporting experiments with cultured human OA cartilage confirmed the ability of paroxetine to mitigate CH and cartilage degradation. Our findings present elevated GRK2 signaling in chondrocytes as a driver of CH in OA, and identify paroxetine as a novel disease-modifying drug for OA treatment.

Introduction

Osteoarthritis is the most prevalent joint disorder in the United States, affecting over 30 million adults nationwide. An estimated 80% of the population will develop radiographic evidence of OA by age 65 (/). As the fifth leading cause of disability, OA is increasingly impacting the personal lives of patients while also placing a significant financial strain on the healthcare system (2-4). OA accounted for over $4000 in annual lost wages per patient, with associated healthcare costs comprising approximately 1% of the US GDP at $305 million in 2013 (5). With no disease modifying treatment available and with the growing prevalence of OA risk factors within the US population, OA will carry an even greater burden in the future (3). Therefore, there is a dire need to identify novel therapeutic targets, approaches and/or agents that can actively halt or reverse the OA disease process.

In OA, articular chondrocytes acquire an aberrant phenotype in which they undergo hypertrophic differentiation (6, 7). Hypertrophic chondrocytes play a central role in OA development and progression by secreting elevated levels of proteolytic enzymes, such as matrix metalloproteinasel3 (MMP13) and aggrecanases, leading to damage and calcification of the articular cartilage (6, 7). Therefore, prevention of articular chondrocyte hypertrophy (CH) is a primary target for therapeutic interventions in OA patients (6, 7).

Chondrocytes respond to a variety of external signals via transmembrane G protein-coupled receptors (GPCRs), the largest family of membrane proteins in the human genome. They mediate physiological responses to hormones, neurotransmitters, inflammatory mediators and environmental stimulants. Generally, agonist stimulation of GPCRs induces intracellular signaling largely through the G-protein Ga subunit. This is normally followed by signal termination, which occurs as a cascade initiated by Gβγ-mediated recruitment of GPCR kinase 2 (GRK2) to the agonist-occupied receptor, followed by GRK2-mediated GPCR phosphorylation leading to its internalization (8-10). We and others have shown that elevated GRK2 levels in heart and kidney disease lead to GPCR desensitization, loss of the physiologic Gα signaling, and, importantly, pathologic cell growth and hypertrophy (11, 12). On the other hand, inhibiting GRK2 membrane recruitment prevents this pathologic cell growth and hypertrophy by recovering the balance in GPCR regulation and signaling (11, 12). Several pharmacologic agents are successfully used to inhibit GRK2-mediated GPCR desensitization, however, none are FDA approved for clinical use (13). Recently, paroxetine, an FDA approved selective serotonin reuptake inhibitor (SSRI), was identified as a potent GRK2 inhibitor with higher selectivity for GRK2 over other GRKs both in vivo and in vitro (14). The pleckstrin homology (PH) domain region of GRK2 is the binding region of GRK2 to Gβγ, paroxetine binds to GRK2 and induces a stabilized conformational change, which inhibits the ability of the PH domain of GRK2 to bind to Gβγ, thus inhibiting GPCR phosphorylation, desensitization and impaired Ga signaling (14). Alternatively, the Gβγ inhibitor gallein is used to indirectly inhibit GRK2-mediated GPCR desensitization (15). Gallein binds to Gβγ and blocks the GRK2 binding spot, thus inhibiting Gβγ-mediated GRK2 membrane recruitment, and the resultant GPCR phosphorylation, desensitization and impaired Gas signaling(/5), as we previously reported in other tissues and in vitro systems (11, 12). The therapeutic efficacy of paroxetine and gallein has been demonstrated in several animal models of heart and kidney disease (10-12, 16, 17).

In chondrocytes, GPCR-Gas signaling prevents CH in the growth plate (18, 19). However, the role of GRK2 in regulating GPCR-Gαs signaling and, thus, CH during OA development and progression is unknown. Here, we hypothesize that in OA GRK2 expression is elevated, which promotes GPCR desensitization leading to the loss of protective Gas signaling, CH and cartilage degeneration. Thus, we investigated the role of GRK2 in CH and cartilage degeneration in OA by using an inducible chondrocyte-specific GRK2 knockout mouse (GRK2-cKO) and a surgical DMM (destabilization of the medial meniscus) model, which is a slowly progressing OA model that resembles the clinical progression of post-traumatic osteoarthritis (PTOA) (20-22). Further, we investigated the therapeutic efficacy of the widely used, GRK2-inhibiting antidepressant paroxetine in OA mice. Mechanistically, we determined the change in GRK2 expression in OA and its effect on GPCR desensitization and Gas signaling by measuring levels of cyclic AMP (cAMP), the Gas second messenger. Finally, we used human cartilage obtained from OA patients to test the translational potential of our findings.

Materials and Methods

Study Design

Sample size was determined using the G*Power 3.1 software. Based on our previous experience and studies, the area of uncalcified tibial cartilage and the total number of chondrocytes are the two main parameters affected by DMM. Based on the difference between these two groups we calculated the sample size required for each group to reach 5% significance and 0.80 power. Power analysis showed that at least 4 mice in each group are required. Thus, we increased the number of mice to 6 in the sham and vehicle groups (both wild type and cKO mice). For the drug treatment groups, we increased the number to 8-9 to account for any unexpected drug induced side effects. In mouse cohorts used for cAMP analysis, tissues from 3 knees were pooled and homogenized to obtain N of 1, with N=5 per group (5% significance and 0.80 power) based on power analysis using preliminary data of Sham and DMM+V groups, thus a total of 15 mice per group were used. Similarly, N=5 was used for the time course study. In human tissue experiments, we collected samples up to N=5/group, and power analysis was performed based on cAMP production under vehicle, paroxetine or gallein treatment, N=5 produced results with 5% significance and 0.80 power. The end point of 12 weeks post-DMM was chosen based on our and others experience with the DMM model where end stage OA develops. The short-term studies, where a 9 weeks-post-DMM end point was performed, were added after the 12 weeks-post-DMM data were obtained. We performed similar power analyses for the short-term studies based on cAMP data obtained from the time course DMM study where N=5 was obtained. The surgeon and lab personnel performing daily drug injections or tamoxifen injections were blinded to the animal identity. All animals and human tissue were randomized for treatment. All mice treatments were initiated after the development of osteoarthritis and all mice and their collected samples and human tissue samples were coded and analyzed in a blinded manner until data were obtained and quantification was completed. Data were then decoded and matched to the corresponding groups, results were charted and statistical analysis was performed. Outliers data determined by Grubb's test were excluded. All results were confirmed by repetition 3 times. cAMP assay and mRNA qRT-PCR were performed in triplicates per sample.

Procurement and Treatment of Human Cartilage

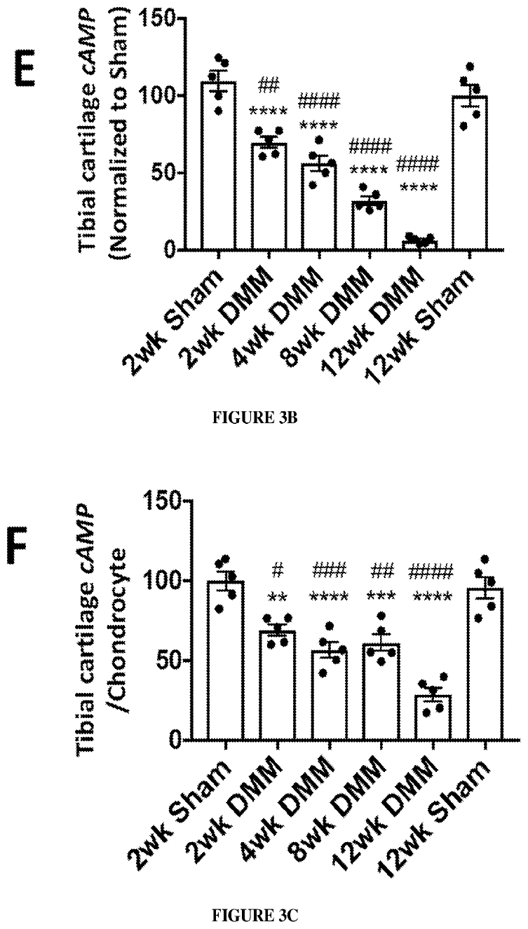

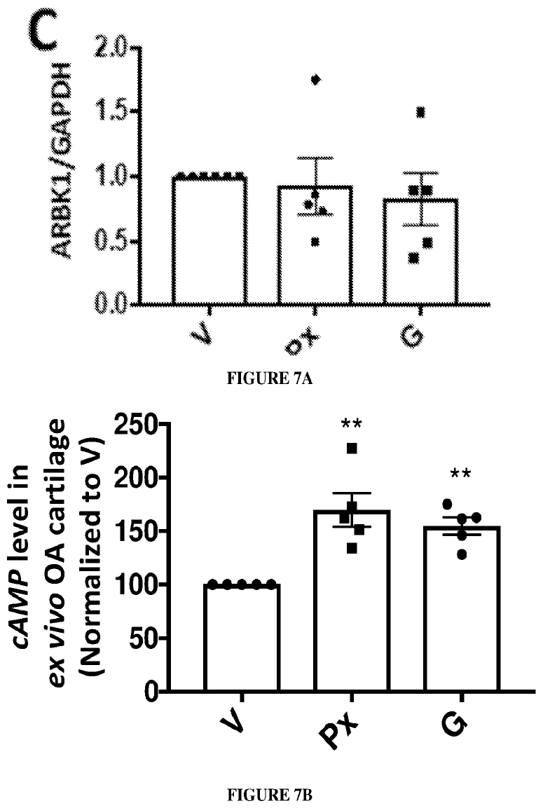

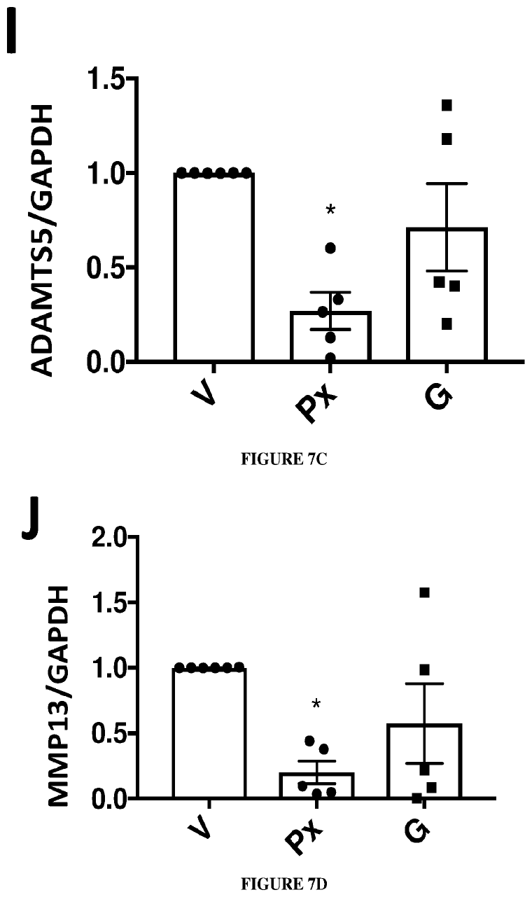

An Institutional Review Board-approved protocol was executed to collect discarded de-identified cartilage from patients. For the experiment described in FIG. 3A, we obtained acutely injured human cartilage (from patients undergoing arthroscopic surgery 4 weeks following meniscal injury, N=6) and OA cartilage (from patients undergoing arthroplasty, N=6). Normal controls were obtained from amputees' knees (N=6). Cartilage was fixed in formalin then processed and embedded in paraffin, 5-μm sections were used for immunofluorescence (IF) staining. For the experiment described in FIG. 7, OA osteochondral tissue was collected from patients undergoing total knee arthroplasty. Osteochondral plugs (8-mm diameter) were obtained and bone was flushed with saline solution to remove all bone marrow and blood cells. Plugs were then cultured overnight in low glucose DMEM at 5% CO₂ and 37° C. The next morning plugs were treated with either vehicle phosphate buffer saline (PBS), paroxetine (5 μM), or gallein (10 μM), and the drug-containing culture medium was replenished after 24 hrs. After 48 hrs, plugs were fixed for 7 days in 10% neutral buffered formalin (NBF), decalcified for 2 weeks in 10% w/v EDTA, embedded in paraffin, and 5-mm sections were cut and mounted for histological and IF staining. Alternatively, the cartilage was shaved from plugs and flash-frozen in liquid nitrogen for RNA extraction as described below.

Animals

Twelve-week-old male C57BL/6J mice were purchased from The Jackson Laboratories. GRK2^{f/f} mice were kindly donated by Dr. Walter Koch at Temple University. Agc1^{tm(IRES-CreERT2)} mice were purchased from the Jackson Laboratory (Jax Laboratory). GRK2^{f/f}/Agc1^{tm(IRES-CreERT2)} mice and their littermate GRK2^{f/f} mice were obtained from male GRK2^{f/f}/Agc1^{tm(IRES-CreERT2)} mice bred with GRK2 f/f female mice, and backcrossed five times. All mice were housed in groups of 3-5 mice per micro-isolator cage in a room with a 12-hour light/dark schedule. All animal procedures were performed according to the National Institute of Health (NIH) Guide for the care and use of laboratory animals and approved by the Animal Care and Use Committee of the University of Rochester and Pennsylvania State University.

DMM surgery is described in the Supplementary Materials section.

Experimental Groups

For all animal studies, we performed two cohorts of mice where in one cohort the knees were collected, formalin fixed and paraffin embedded. In the second cohort, cartilage from 3 mice was pooled and was used to study Gas activity by measuring cAMP production.

DMM Time-course Study: Twelve-week old male wild type C57BL/6 mice received DMM surgeries and were sacrificed 2, 4, 8, or 12 weeks later. Sham operated mice were harvested either 2 or 12 weeks following sham surgeries.

Chondrocyte-specific GRK2 gene deletion study: Twelve-week-old littermate male GRK2^{f/f}/Agc1^{tm(GRES-CreERT2)} mice had DMM surgery as described above to induce PTOA. Tamoxifen (in corn oil) was administered in three consecutive ip injections (1.5 mg/10 g mouse weight) 7 weeks after surgery to achieve chondrocyte specific GRK2 deletion 8 weeks after DMM (where OA is in place), in order to determine the role of articular chondrocyte GRK2 signaling in the progression of OA. Littermate GRK2^{f/f} mice receiving the same course of tamoxifen injections were used as non-KO controls. Mice were sacrificed 8 weeks post DMM, at 20 weeks of age, to confirm GRK2 deletion; or 12 weeks post DMM surgery, at 24 weeks of age, for analysis of OA progression.

Drug treatment studies: In twelve-week old male wild type C57BL/6 mice that received DMM surgeries, vehicle (PBS), paroxetine (5 mg/kg/day), fluoxetine (5 mg/kg/day), gallein (10 mg/kg/day) or indomethacin (2.5 mg/kg/day) were administered daily by ip injection. Drug treatment was initiated 8 weeks following DMM to determine the role of GRK2 signaling in the progression of OA.

Short term chondrocyte-specific GRK2 gene deletion study: To evaluate the acute effect of GRK2 deletion in articular chondrocytes, GRK2^{f/f} or GRK2^{f/f}/Agc1^{tm(IRES-CreERT2)} mice received sham or DMM surgery followed by tamoxifen injections at 7 weeks post DMM to achieve GRK2 conditional deletion 8 weeks post DMM. Mice were sacrificed 9 weeks post DMM, 21 weeks of age, for analysis.

Short term drug treatment study: To evaluate the acute effect of drug treatments, GRK2^{f/f} mice received sham or DMM surgery followed by treatment with vehicle, paroxetine, gallein, fluoxetine, or indomethacin with the doses indicated above for 1 week beginning at 8 weeks post DMM. All mice were sacrificed 9 weeks post DMM, 21 weeks of age, for analysis of OA progression.

OARSI scoring of cartilage, Histomorphometry—(Safranin-O/Fast Green) coupled with histomorphometry using the Osteomeasure® system, IF staining, cAMP assay, and Micro-CT assessment are detailed in Supplementary Materials.

RNA Purification and Real Time-Quantitative Polymerase Chain Reaction (RT-qPCR)

mRNA was isolated from human cartilage using the method we published recently, yielding RNA Integrity Number (RIN) values above 7.0 (53). cDNA was prepared using Iscript cDNA synthesis kit from Bio-rad following the manufacturer's protocol. cDNAs were qPCR amplified using TaqMan Gene Expression Assays, with GAPDH as the house keeping gene (ThermoFisher Scientific; described in Table 2) and the 7500 Fast Real-Time PCR System (Applied Biosystems).

TABLE 2
List of the gene expression assays used for RT-qPCR.

| | Gene Expression |
|---|---|
| Gene Name | Assay Cat. No |
| Human MMP13 | Hs00942584 |
| Human ADAMTS5 | Hs00199841 |
| Human ARBK1 (GRK2) | Hs00176395 |
| Human GAPDH | Hs02758991 |

Statistical Analyses

Multiple responses of various physiological and biochemical assays were analyzed using unpaired t-test or one-way ANOVA as indicated in mouse studies, and paired t-test for human cartilage experiments. For ANOVA, Tukey's post-hoc analysis was performed if statistical significance (P<0.05) was achieved. All calculations were performed using the GraphPad Prism 7.0 program.

Destabilization of the Medial Meniscus (DMM) Surgery

Twelve-week-old male mice were administered DMM surgery to the right knee and sham surgery to the left knee as described (S. S. Glasson, Osteoarthritis. Cartilage. 15, 1061-1069 (2007)). Briefly, mice were anesthetized via intraperitoneal injection of ketamine (60 mg/kg) and xylazine (4 mg/kg), and a 5-mm-long incision was made on the medial side of the knee. Under a dissecting microscope, an incision was made along the medial side of the patellar tendon, opening the joint space. Using a #11 scalpel, the medial meniscotibial ligament (MMTL) was transected, enabling the medial meniscus to move freely. A similar skin incision was made in sham knees, but the joint structure was not disturbed. For the sham group, both right and left knees had sham surgeries. After surgery, 4-0 silk sutures were used to close the incision using an interrupted pattern. Mice were provided analgesia via intraperitoneal injection of buprenorphine (0.5 mg/kg) every 12 hours for 72 hours, and sutures were removed after 7 days. Mice were sacrificed, at the indicated time points, by anesthesia followed with whole animal perfusion using 10% NBF, knees were harvested and fixed for 7 days in 10% NBF, decalcified for 7 days in 10% w/v EDTA, embedded in paraffin, and 10-μm sections were cut and mounted for Safranin-O/Fast Green or IF staining.

OARSI Scoring of Cartilage

Semi-quantitative histopathologic grading was performed using a derivative of the Chambers' scoring system [74, 83] that has been established by the OARSI histopathology initiative as the standard method for grading of mouse cartilage degeneration [84]. Based on this system, cartilage grading was carried out using Safranin-O/Fast Green-stained midsagittal sections. Three sections from representative levels (50 μm apart) of the medial compartment of the joint were selected for each sample and, which were evaluated in a randomized, blinded manner by 3 laboratory members. The 3 scores were averaged to calculate the section score, and the sample score was then calculated by averaging the scores obtained from the three levels. Grading was performed using the following scale: 0=normal cartilage, 0.5=loss of proteoglycan stain without cartilage damage, 1=mild superficial fibrillation, 2=fibrillation and/or clefting extending below the superficial zone, 3=mild (<25%) loss of cartilage, 4=moderate (25-50%) loss of cartilage, 5=severe (50-75%) loss of uncalcified cartilage, and 6=eburnation with >75% loss of cartilage. Grading was performed by three blinded observers (F.K., M.J.Z., and E.R.S.). Observer agreement was evaluated in pairs via calculation of a weighted kappa coefficient, using Fleiss-Cohen weights, as we have described [38]. The F.K. versus M.J.Z. coefficient was 0.92, the F.K. versus E.R.S. coefficient was 0.94, and the M.J.Z. versus E.R.S coefficient was 0.89, all indicative of strong agreement between the observers.

Histomorphometry—(Safranin-O/Fast Green) Coupled with Histomorphometry Using the Osteomeasure® System

Using Safranin-O/Fast Green-stained sections, the OsteoMetrics system was used to quantify the above parameters on three sections from representative levels (50 μm apart) of the medial compartment of the joint for each sample. Live images of the center of the knee joint were collected through an Olympus microscope (10× objective) outfitted with a camera, a stylus was used to trace the regions of interest (ROIs). A blinded observer quantified uncalcified articular cartilage area, total chondrocyte number and matrix-producing chondrocyte number using the built-in area calculation algorithms and quantification functions of the OsteoMeasure® system. Notably, the same Safranin-O and Fast Green stained sections used in OARSI scoring (see above) were used for OsteoMeasure® analysis as detailed in our recent publication (W. J. Pinamont, “Standardized Histomorphometric Evaluation of Osteoarthritis in a Surgical Mouse Model.” J. Vis. Exp. In press, (2020)). Briefly, the total cartilage area was measured by the first line was drawn across the superior edge of the cartilage surface where the cartilage meets the joint space. A second line was drawn at the chondro-osseous junction where the calcified cartilage meets the subchondral bone. The calcified cartilage was determined with a line drawn along the tide mark, which is the naturally occurring line separating the calcified and uncalcified regions of the articular cartilage. A second line was drawn at the chondro-osseous junction where the calcified cartilage meets the subchondral bone. The uncalcified cartilage area was determined by subtracting the calcified cartilage area from the total area of cartilage. Total chondrocyte number and matrix-producing chondrocyte number were counted within the uncalcified cartilage region using count functions within the histomorphometry system. Matrix-producing chondrocytes were counted based on the region of Safranin-O staining within the extracellular matrix surrounding the articular chondrocyte, indicating anabolic signaling to maintain matrix homeostasis. Similarly, the anterior femoral synovial thickness was measured using OsteoMeasure® software. Synovial membrane thickness extending from anterior horn of the medial meniscus to the femur was determined by drawing a line from the inner insertion point on the femur towards the attachment on the meniscus. A second line was drawn from the outer insertion of the femur towards the attachment to the meniscus. The synovial thickness was calculated by dividing total synovial area by synovial perimeter.

IF Staining

Paraffin sections were deparaffinized in three changes of xylene for five minutes each, rehydrated in ethanol (two changes of 100% ethanol, followed by two changes of 95% ethanol, followed by one change of 70% ethanol) and rinsed twice in deionized water. Antigen retrieval was performed for 30 minutes at 37° C. using 0.4% pepsin (Sigma P-7000) in 0.1M hydrochloric acid (HCl) and was followed by permeabilization for 30 minutes at room temperature using Triton X in tris buffered saline (TBS). Sections were then blocked for 1 hour at room temperature in 10% normal goat serum in 1×TBS. Tissue sections were incubated overnight at 4° C. with the rabbit anti-mouse primary antibody specific for the target protein (detailed in Table 1).

TABLE 1
List of the primary antibodies (ab, from Abcam and QL, from
Thermo fisher) used for immunofluorescent staining.

| Protein Name | Antibody Cat. No |
|---|---|
| GRK2 | ab137666 |
| MMP-13 | ab39012 |
| ADAMTS-5 | ab41037 |
| Aggrecan | ab3778 |
| Aggrecan-Neo | QL229484 |

Following three 5-minute washes in 1×TBS, slides were incubated for 1 hour at room temperature with biotinylated goat anti-rabbit secondary antibody (Life Technologies, Waltham, MA, USA), washed again with 3 changes of 1×TBS and incubated for 1 hour at room temperature with Alexa fluor 647 Streptavidin. Finally, slides were washed with three changes of 1×TBS for 5 minutes each, followed by one wash with 1×TBS with tween 20 (TBST), and a final 5-minute wash in 1×TBS. Mounting and nuclear staining was performed using ProLong™ Gold antifade reagent with DAPI (Invitrogen, Waltham, MA, USA).

cAMP Assay

Gas activity was measured by quantifying cAMP content in cartilage pooled from 3 mice. Tibial cartilage was dissected out and snap frozen in liquid Nitrogen. As positive control, tibial cartilage obtained from normal mice was treated with 0.5 mM 3-isobutyl-1-methylxanthine (IBMX) for 30 minutes, followed by 0.1 mM Forskolin for 15 minutes, then snap frozen in liquid Nitrogen. For human cartilage cAMP content, 70 mg frozen cartilage were used. Frozen cartilage was pulverized using 500 μL lysis buffer as we have previously described (H. K. Le Bleu, Anal Biochem, 518, 134-138 (2017)). cAMP content was quantified using the Cyclic AMP XP Assay kit (Cell Signaling, #4339) and following manufacturer's protocol, with 0.5 mM IBMX added to the lysis buffer to prevent phosphodiesterase mediated cAMP degradation during the extraction process. cAMP levels in different groups were normalized to that of sham mice or the vehicle treated human cartilage.

Micro-Computed Tomography (Micro-CT) Assessment

Prior to histologic processing, harvested knee joints were evaluated via micro-CT using a Scanco vivaCT40 scanner with a 55 kVp source as we have previously described [38]. Joints were scanned at a resolution of 10.5 μm. General 3-dimensional images were obtained with simple segmentation at a Scanco threshold of 260, which was determined on inspection of the first specimen. This translates to a linear attenuation coefficient of 2.080 cm-1. Knee analysis starts by locating the highest section of the tibial plateau and defining that as the center point. All bone is captured 100 slices in each direction from the center point. The patella and fibula, if present in that region, are excluded. The fabellae are considered part of the knee and are included. A threshold of 260 (2.080 cm′) is used to analyze bone volume and microarchitecture. Tibia subchondral analysis begins by starting at the knee and proceeding distally until the cortical shell is penetrated. All non-cortical bone is included, both solid and trabecular. Osteophytes were identified as protrusions and counted using cross-sectional images of the 3-dimensional stacks. Subchondral plate thickness was calculated by determining the number of cross-sections forming the subchondral plate and multiplying it by section thickness (C. Huesa, Annals of the rheumatic diseases, 75, 1989-1997 (2016)).

Results:

GRK2 expression is elevated with reduced cAMP levels in clinical and preclinical OA. To investigate the involvement of GRK2 in OA, we first analyzed its expression levels in normal and injured human cartilage using immunofluorescence (IF) staining. Data indicated pathologically elevated GRK2 expression in injured human cartilage, both in acute (meniscal injury) and chronic OA disease stages. Next, we performed the same analysis on knee cartilage collected from mice following sham or DMM surgery to induce PTOA that resembles clinical OA in its development and progression (20-22) (FIG. 3A). GRK2 expression increased significantly during the early stages of OA, and further increased in a time-dependent manner as OA progressed to late stages.

cAMP is the Gas second messenger, therefore, activation of Gas enhances cAMP production (9). Thus, GRK2-mediated GPCR desensitization leads to decreased Gas-cAMP signaling (8, 9). To corroborate our results of enhanced GRK2 expression in OA, we measured the level of cAMP in cartilage from sham and DMM mice. As expected, we detected significant reductions in the level of cAMP in DMM as compared to sham cartilage as early as 2 weeks post-surgery, and the level of cAMP was further reduced as OA progressed (FIG. 3B, C). Collectively, these data indicate that induction of OA results in an immediate and profound increase in GRK2 expression resulting in significant inhibition of Gas signaling, which is further intensified as OA progresses to late stages. These data suggest a role for GRK2-mediated GPCR desensitization in OA development as well as progression.

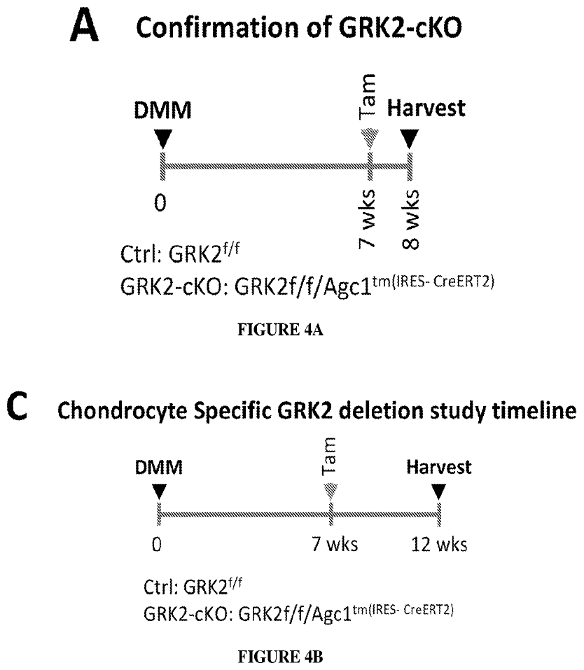

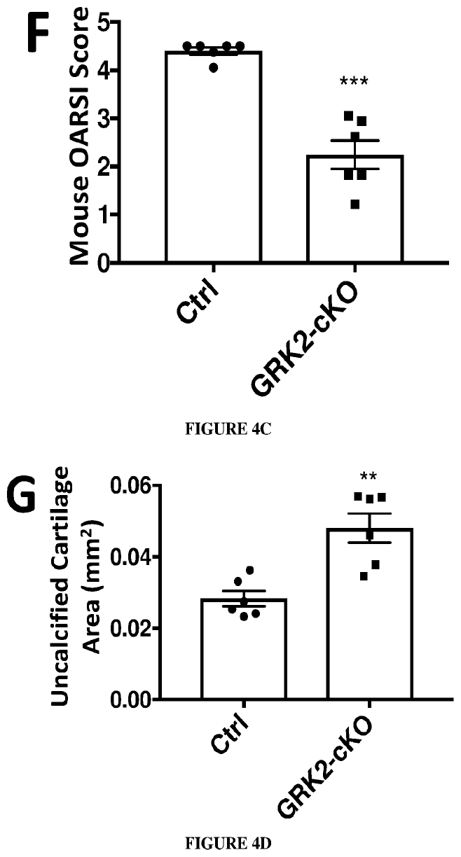

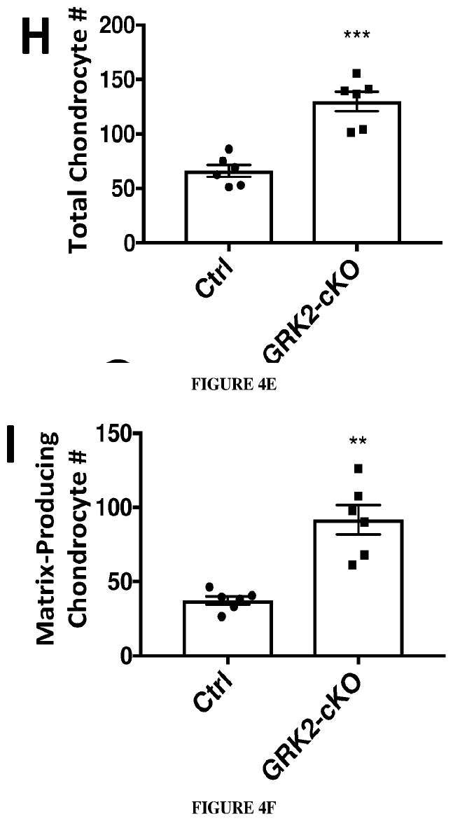

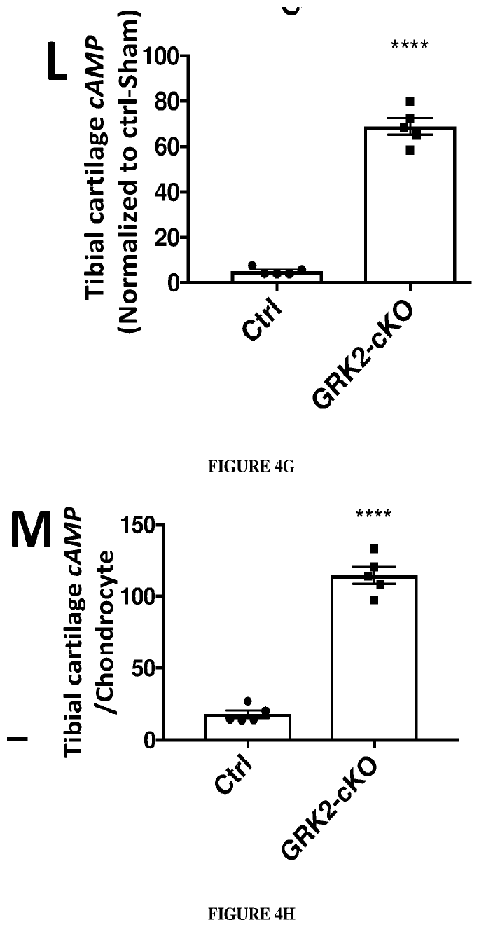

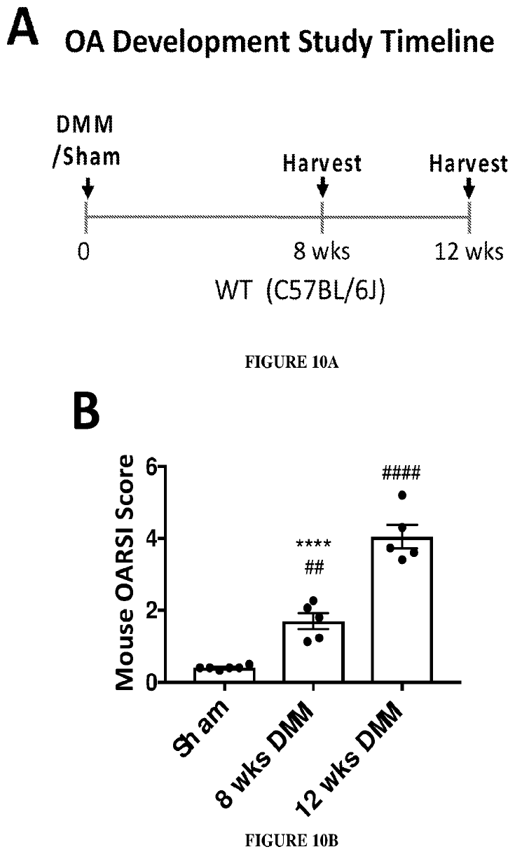

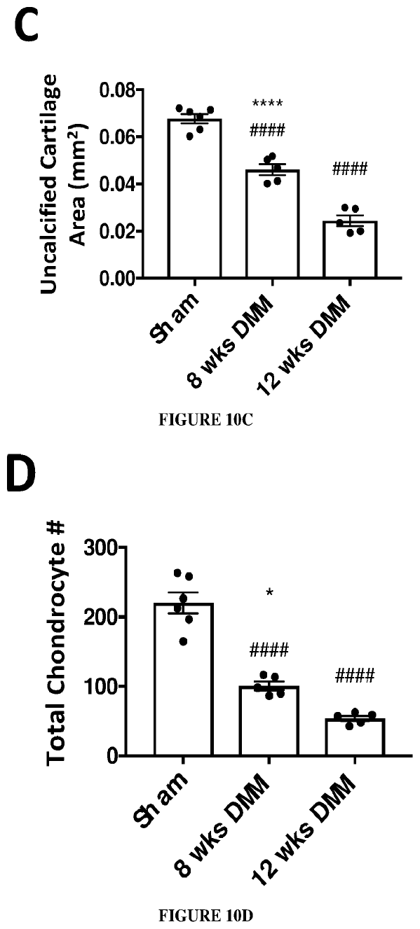

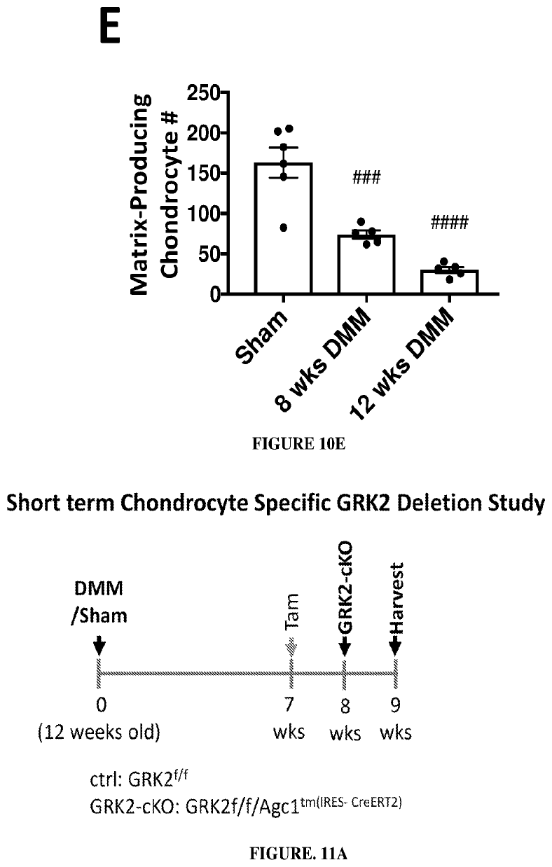

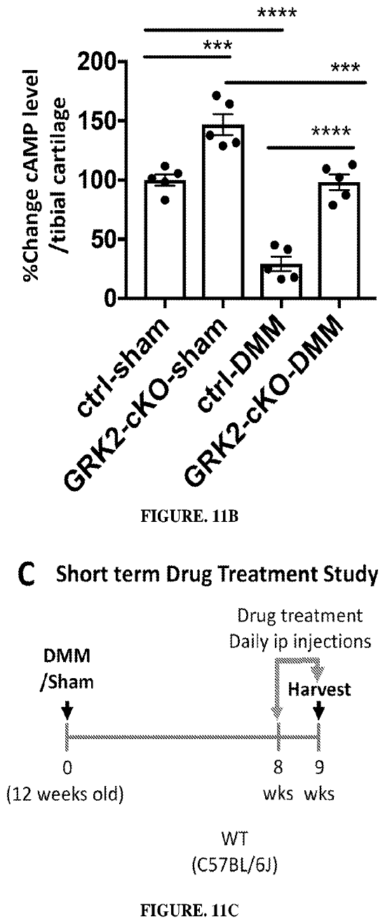

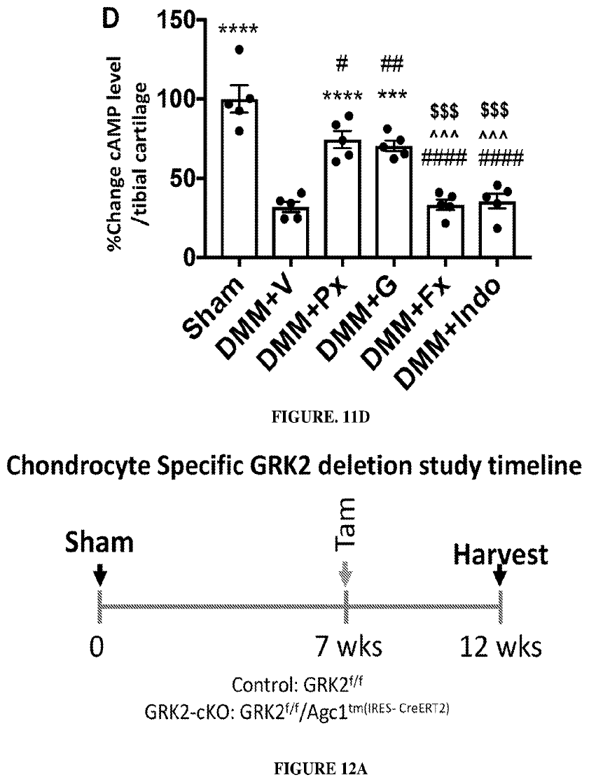

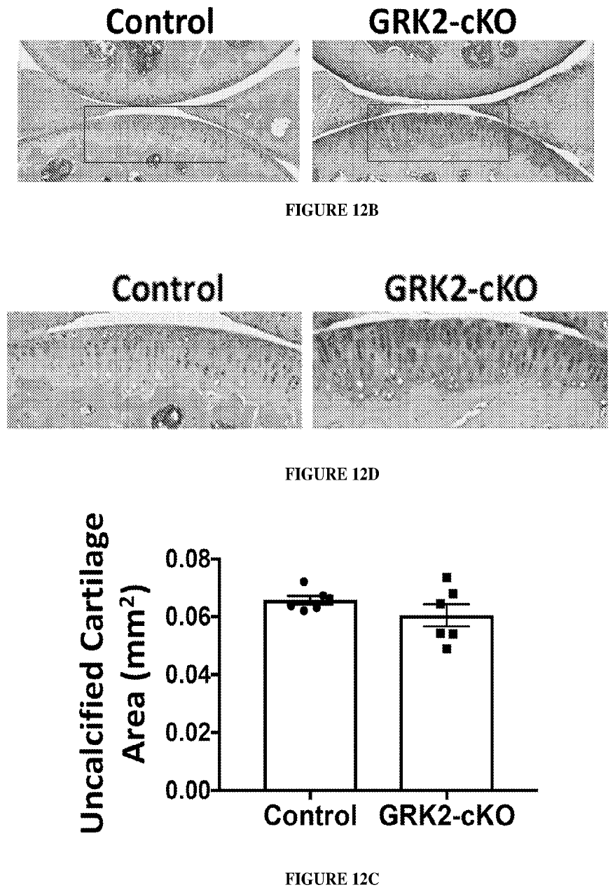

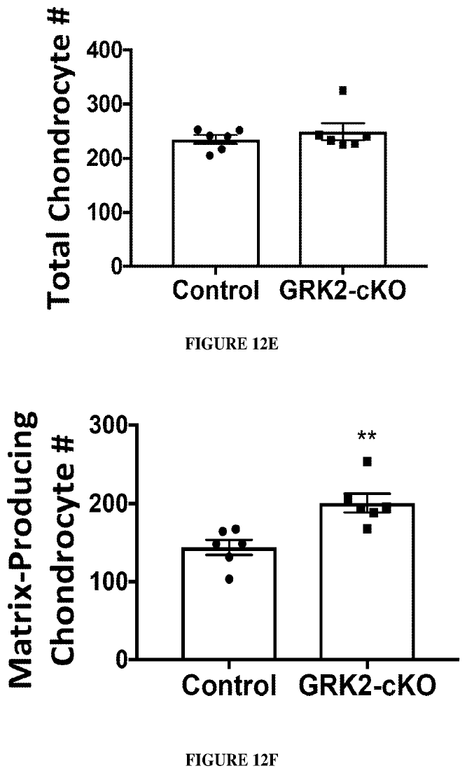

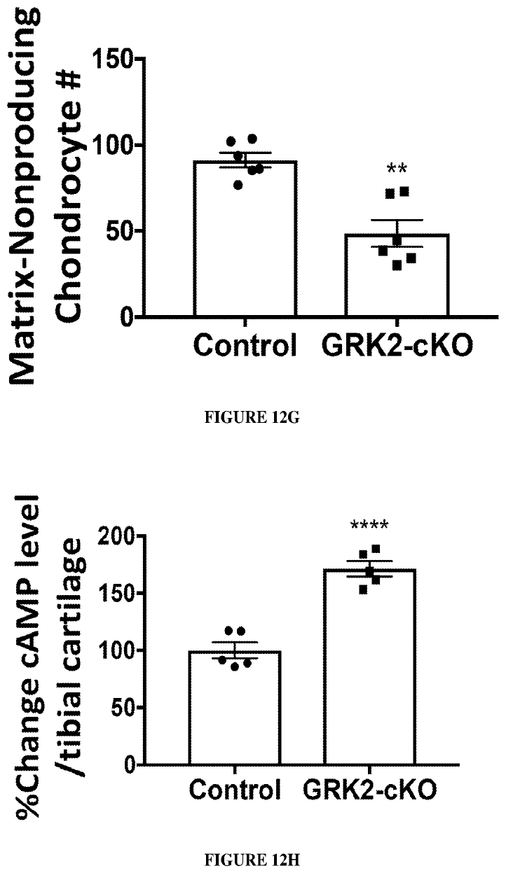

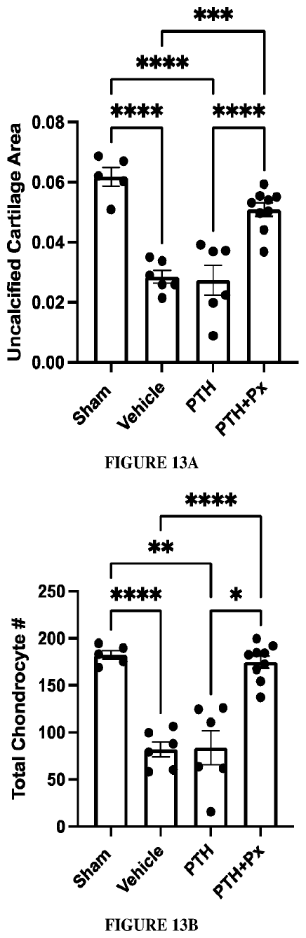

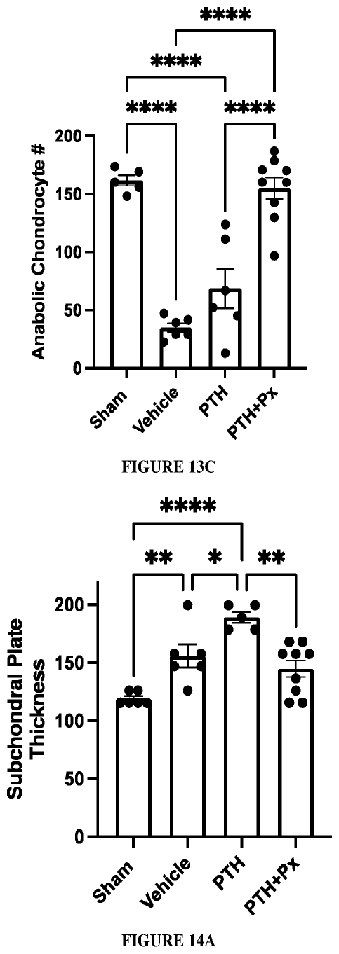

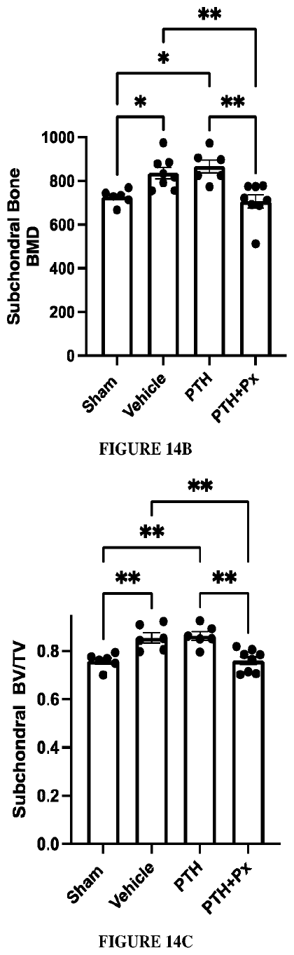

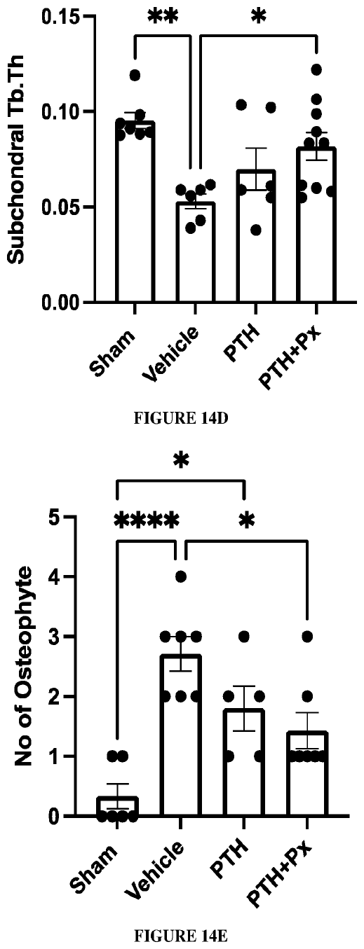

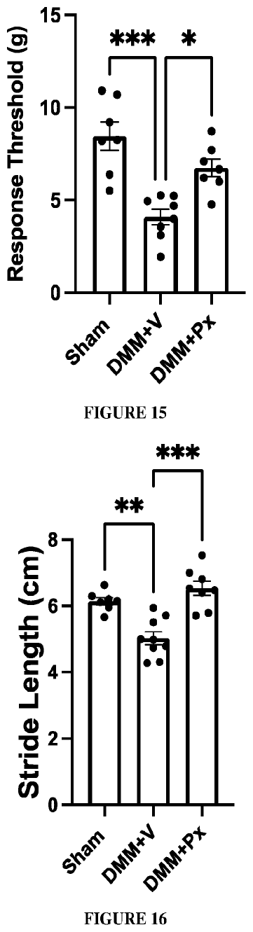

Cartilage specific GRK2 deletion normalizes chondrocyte Gas signaling, decelerates OA progression, and promotes matrix regeneration. Clinically, patients diagnosed with OA present with an actively progressing disease state, where there is some but incomplete cartilage degeneration and chondrocyte loss. To determine the role of GRK2 in OA progression and its validity as a novel therapeutic target, we used a clinically relevant PTOA mouse model (DMM); since meniscal injury leading to joint disability is identified as one of the major underlying causes of OA (20, 22). Surgical DMM is widely accepted as a reproducible slowly progressing PTOA model, where chondrocyte hypertrophy and loss is identified as the central mechanism for cartilage loss and disease progression in human OA and is replicated in DMM joints (20-22). We induced chondrocyte-specific GRK2 deletion (GRK2-cK0) in mice with progressing OA, 8 weeks following DMM, which represents clinically progressive OA that is not end stage yet, where there is little cartilage degeneration but significant chondrocyte loss and hypertrophy (20) (FIG. 10). The Agc1^{tm(IRES-CreERT2)} mouse was used to specifically target articular chondrocytes in adult cartilage (23) (see materials and methods). Tamoxifen was injected at week 7 post-DMM to the GRK2^{f/f}/Agc1^{tm(IRES-CreERT2)} mice (GRK2-cK0) and their littermate GRK2^{f/f} mice as the non-knockout control, to induce GRK2 deletion at week 8 post-DMM. Mice were then divided into two groups, the first group was harvested at week 8 and used to confirm GRK2 deletion (FIG. 4A), which is reported to take place 3 days following the last tamoxifen dose (23). Based on GRK2 IF staining in the tibial articular cartilage in control and GRK2-cKO mice harvested, GRK2 protein expression was lost in the uncalcified cartilage chondrocytes, but importantly, persisted in the calcified cartilage chondrocytes and in the subchondral region, in agreement with the reported phenotype of the Agc1^{tm(IRES-CreERT2)} mice (23). The second group was sacrificed 12 weeks post-DMM to study the impact of chondrocyte GRK2 deletion on OA progression (FIG. 4B). Knee joints harvested from control and GRK2-cKO mice were safranin-O and Fast Green-stained. As compared to control mice, GRK2-cKO mice demonstrated reduced cartilage degeneration and OARSI score (FIG. 4C), increased area of uncalcified cartilage (FIG. 4D), and reduced chondrocyte loss (FIG. 4E). In addition, there was an increase in the number of matrix producing chondrocytes (FIG. 4F) suggesting an anabolic matrix-regenerative effect of GRK2 deletion. Consistent with this, aggrecan IF staining in the whole tibial articular surface demonstrated increased aggrecan expression in the cartilage of GRK2-cKO mice compared to control mice. Furthermore, cAMP production was enhanced in GRK2-cK0-mouse articular chondrocytes (FIGS. 4G & H), suggesting enhanced Gas signaling and reduced GPCR desensitization. However, the loss of cAMP levels 12 weeks post-DMM can be attributed to the loss of chondrocytes. To address this, we analyzed cAMP levels as early as one week following GRK2 deletion i.e. 9 weeks post-DMM (FIGS. 10A & B), a relatively short period to affect chondrocyte loss in DMM mice, where we also found enhanced expression of cAMP. This suggests that following GRK2 deletion, there is an immediate attenuation of GPCR desensitization and recovery of Gas signaling. Similarly, in the sham-operated contralateral knees, GRK2 deletion caused a shift towards anabolic chondrocyte activity with elevated number of matrix producing chondrocytes (FIG. 10F) and elevated articular-cartilage cAMP levels (FIG. 10H). The protective effect demonstrated by GRK2 conditional deletion suggests a main role for GRK2-mediated GPCR desensitization in cartilage degeneration and loss of chondrocyte anabolic activity during PTOA progression, and presents GRK2 as a novel therapeutic target for OA.

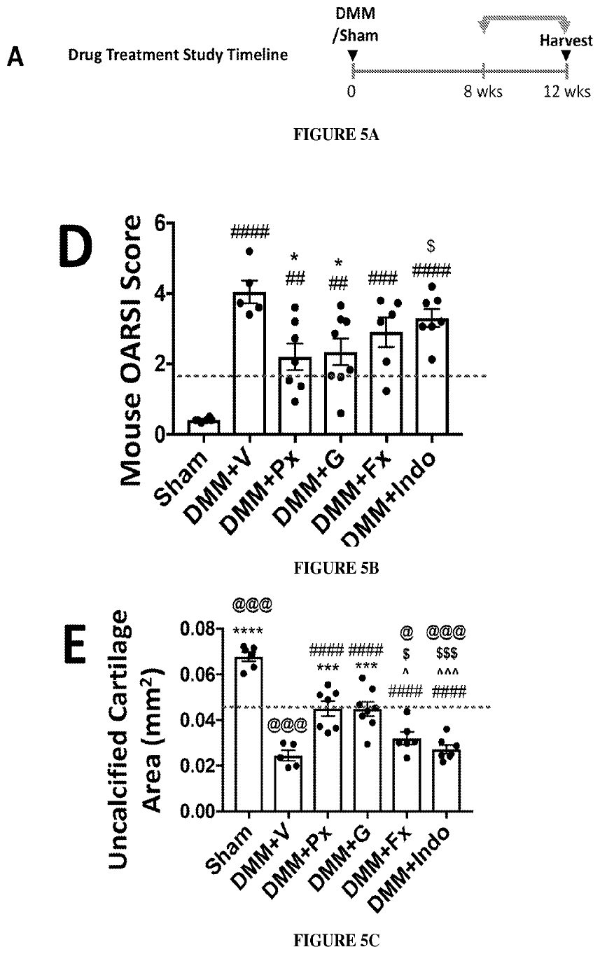

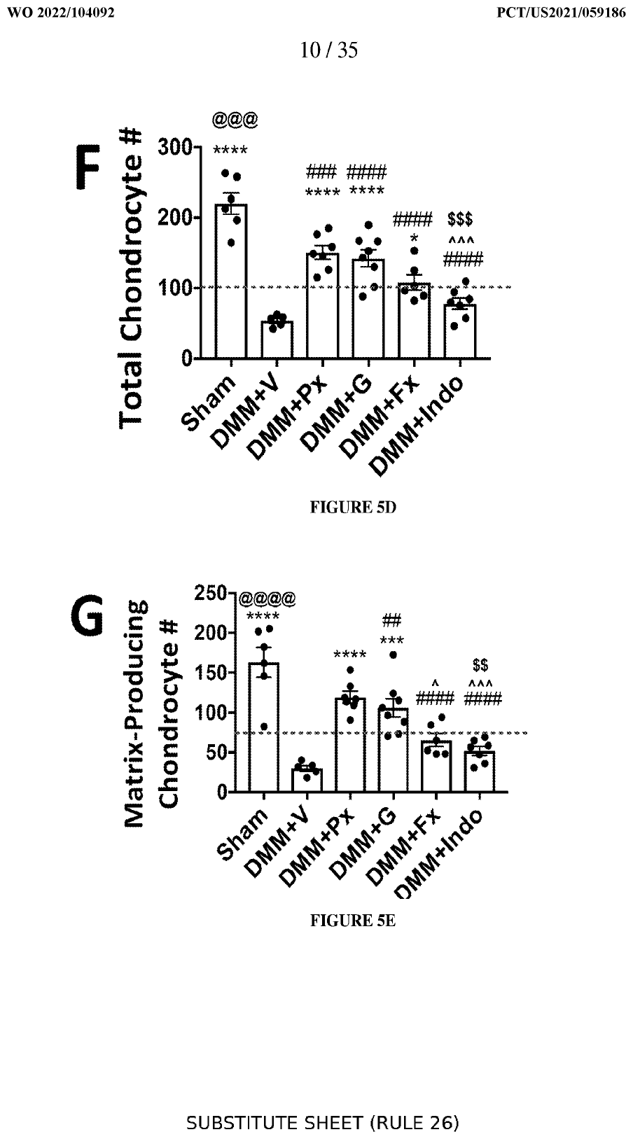

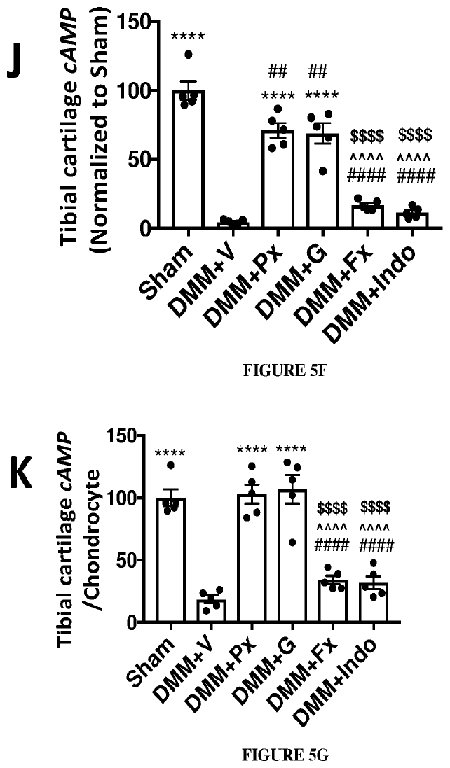

Paroxetine-mediated GRK2 inhibition normalizes chondrocyte cAMP levels, decelerates OA progression and promotes an anabolic chondrocyte phenotype. To determine whether pharmacologic GRK2 inhibition can prevent or decelerate OA progression in the DMM model, we used paroxetine, an SSRI with a direct and selective GRK2 inhibitory effect (24). Alternatively, we used the gallein, a Gβγ inhibitor that blocks the binding spot of GRK2 on Gβγ and thus inhibits its recruitment to the cell membrane and the resultant GPCR desensitization. Drug treatment began 8 weeks after DMM and continued until week 12 (Figure Histological assessment of Safranin-O and Fast Green stained knee joints revealed reduced cartilage degeneration and lower OARSI scores in DMM mice treated with paroxetine or gallein in comparison to vehicle treated mice (FIG. 5D). Further, histomorphometric analysis demonstrated that pharmacologic GRK2 inhibition preserved the uncalcified cartilage area (FIG. 5E) and the total number of chondrocytes (FIG. 5F), indicating a chondro-protective effect, as no deterioration was observed in either parameter from the point of treatment initiation (8 weeks post-DMM, black dotted line) until harvest time (12 weeks post-DMM). In addition, there was a higher number of the matrix producing chondrocytes (Figure as well as enhanced aggrecan expression in paroxetine and gallein treated mice as compared to the vehicle treated group demonstrating enhanced anabolic signaling as shown by aggrecan IF staining in the whole tibial articular surface. Staining was performed in sham and DMM mice receiving the indicated drug treatments as described in (FIG. 5A). Thus, paroxetine and gallein exert both a chondro-protective and a matrix-regenerative effects in DMM mice. Mechanistically, GRK2 inhibition restored cAMP levels in the articular cartilage of paroxetine and gallein treated mice, an effect that was also observed as early as 7 days following drug treatment (FIGS. 10C & D), indicating stimulation of Ga signaling even before chondrocytes are significantly lost. To determine whether the SSRI effect of paroxetine contributes to its therapeutic effect in OA, we treated DMM mice with fluoxetine, an SSRI that lacks any GRK2-inhibitory effects (17, 24). Fluoxetine treated mice showed no preservation of the uncalcified cartilage (Figure SE), with high OARSI score (FIG. 5D) and low matrix-producing chondrocytes number (FIG. 5G). Similarly, cAMP production in the articular cartilage was not different from vehicle treated mice (FIGS. 5J & K). There was only mild preservation of the total chondrocytes number (FIG. 5F) as compared to vehicle treated mice, where they were maintained at similar levels to those at 8 weeks post-DMM. Taken together, these data demonstrate that fluoxetine lacks chondro-protective and matrix-regenerative effects. Accordingly, the therapeutic effects of paroxetine are exerted largely through its GRK2-inhibitory rather than the SSRI effects.

Furthermore, we compared the therapeutic effects of paroxetine and gallein in DMM mice to that of indomethacin, a standard anti-inflammatory drug for pain management in OA (25-27). Indomethacin treatment exhibited no chondro-protective or matrix-regenerative effects in DMM mice (FIG. 5D-G, J, K), indicating that an anti-inflammatory effect does not rescue chondrocyte pathology and cartilage degeneration. Finally, it is important to note that there was progressive cartilage degeneration in the fluoxetine and indomethacin treated groups, as the uncalcified cartilage area was significantly different from that at 8 weeks post-DMM (red-dotted line).

Paroxetine treatment inhibits chondrocyte hypertrophy in MOM OA model. To further investigate the effect of GRK2 inhibition on the pathological signaling in OA chondrocytes, we performed IF staining for the well-established chondrocyte hypertrophy markers a disintegrin and metalloproteinase with thrombospondin motif 5 (ADAMTS5) and MMP13, and for the matrix degradation marker aggrecan neo-epitope (Aggrecan Neo; an aggrecan breakdown product). IF staining images were collected of ADAMTS5, MMP13, Aggrecan Neo epitope and GRK2 in the tibial articular cartilage of sham and DMM mice harvested 12 weeks following surgery. Mice were treated with vehicle (V), gallein (G) or paroxetine (Px) daily following the timeline outlined in FIG. 5A. Examination of the staining in the uncalcified cartilage region indicated, as expected, elevated expression of ADAMTS5, MMP13, and Aggrecan Neo in vehicle-treated DMM mice compared to sham control, signifying CH and matrix degradation that typify OA progression. Importantly, the expression of the three markers were significantly decreased in mice treated with either paroxetine or gallein. These results indicate that GRK2 inhibition attenuates chondrocyte hypertrophy and cartilage matrix degradation and, thus, prevents OA progression. Interestingly, the expression of GRK2 correlated with that of ADAMTS5 and MMP13, suggesting GRK2 as a novel CH marker.

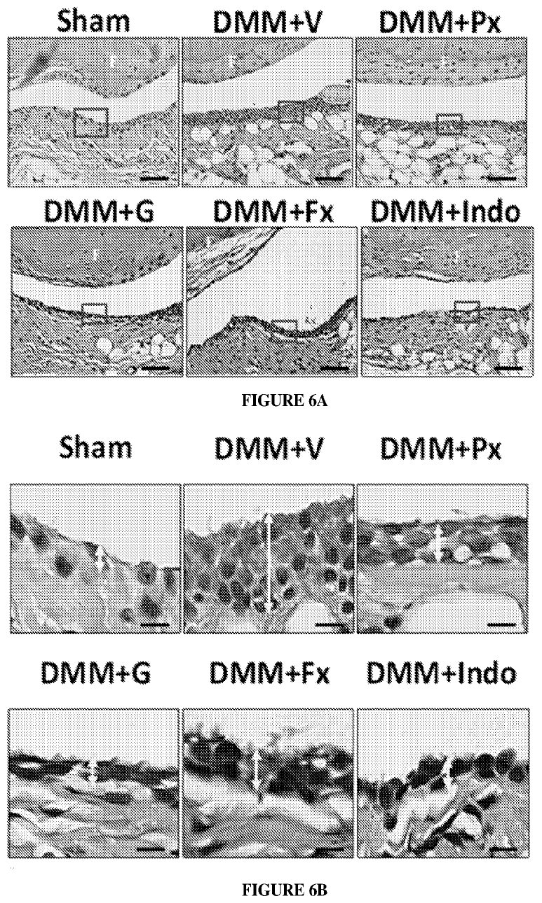

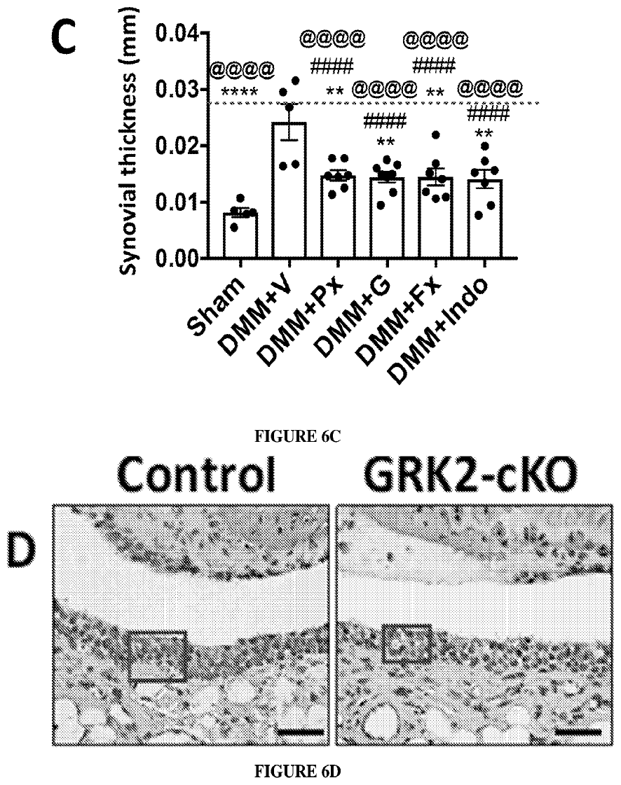

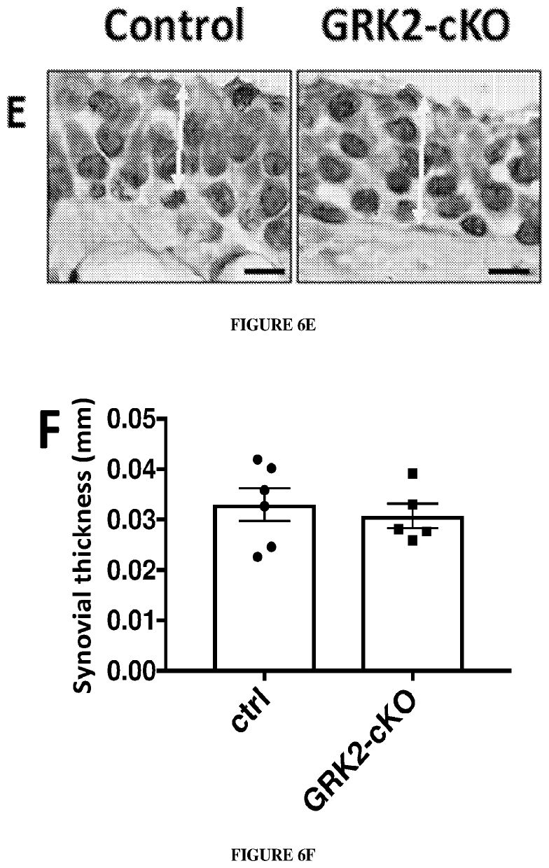

Systemic GRK2 inhibition, but not chondrocyte-specific GRK2 deletion, inhibits synovitis in OA. Synovitis, inflammation of the synovium characterized by synovial membrane thickening is characteristic of OA (28-30). Increased inflammatory cell infiltration to the synovium leads to synovitis and increased production of inflammatory mediators that trigger chondrocyte inflammatory and hypertrophic signaling and inhibit its anabolic signaling (28-31). Following DMM surgeries, synovitis peaks in early stages then declines, but persists at levels higher than those in sham operated mice throughout mid and late stages of PTOA (31). Our data shows that treating DMM mice with the nonsteroidal anti-inflammatory drug (NSAID) indomethacin significantly reduced the synovial membrane thickness as compared to vehicle-treated DMM mice (FIG. 6A-C). Interestingly, paroxetine and gallein were as effective as indomethacin in reducing synovial membrane thickening (FIG. 6A-C). In spite of it is mild anti-OA effect, fluoxetine also attenuated synovial thickening in DMM mice (FIG. 6A-C), in agreement with its previously reported anti-inflammatory effect (32). In contrast to paroxetine and gallein treatment, chondrocyte specific GRK2 deletion in GRK2-cKO mice had no effect on synovial thickness (FIG. 6D-F), although it decelerated OA progression (FIG. 4), which suggest that chondro-protection does not convey a protective effect in the synovium. Furthermore, these data demonstrate that paroxetine- and gallein-mediated GRK2 inhibition attenuates synovitis through direct effects on the synovium that are independent of their chondro-protective effects.

Paroxetine inhibits chondrocyte hypertrophy and promotes cAMP production in human OA cartilage. To investigate the efficacy of pharmacological inhibitors of GRK2 in clinical OA, we examined the effect of paroxetine and gallein on ex vivo cultured human osteochondral plugs obtained from OA patients. Treatment of osteochondral plugs with paroxetine or gallein reduced GRK2 protein expression based on GRK2 IF staining images, while GRK2 mRNA expression was not affected (FIG. 7A). Both treatments were also associated with a significant increase in cAMP production (FIG. 7B). Further, paroxetine treatment significantly inhibited the expression of the cartilage-matrix degrading enzymes ADAMTS5 and MMP13 on both protein (based on ADAMTS5, and MMP13 IF staining images) and mRNA levels (FIGS. 7C & D). Gallein also attenuated ADAMTS5 and MMP13 protein expression (based on ADAMTS5, and MMP13 IF staining images). The impact of gallein on the mRNA expression of ADAMTS5 and MMP13, although did not achieve statistical significance, showed a similar trend to paroxetine (6C & D). Therefore, GRK2 pharmacological inhibition by paroxetine exerts a direct chondro-protective effect in human OA cartilage.

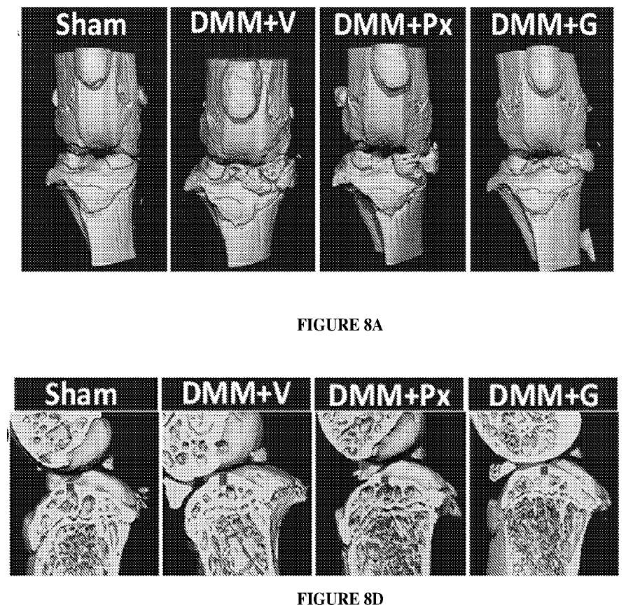

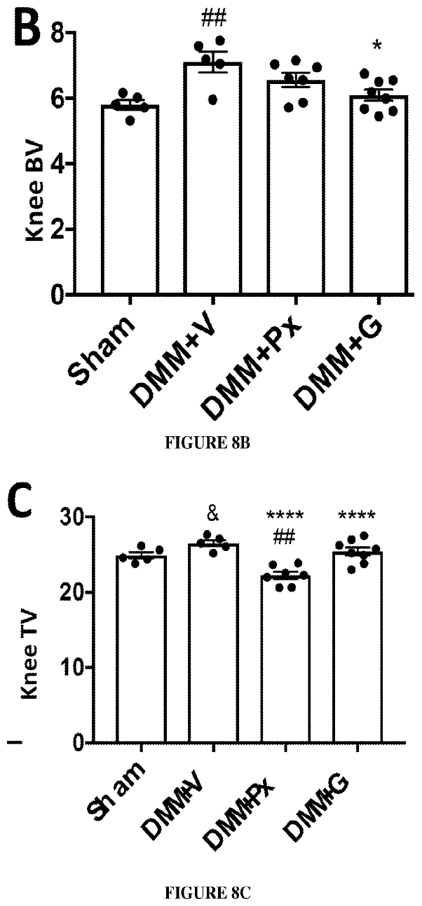

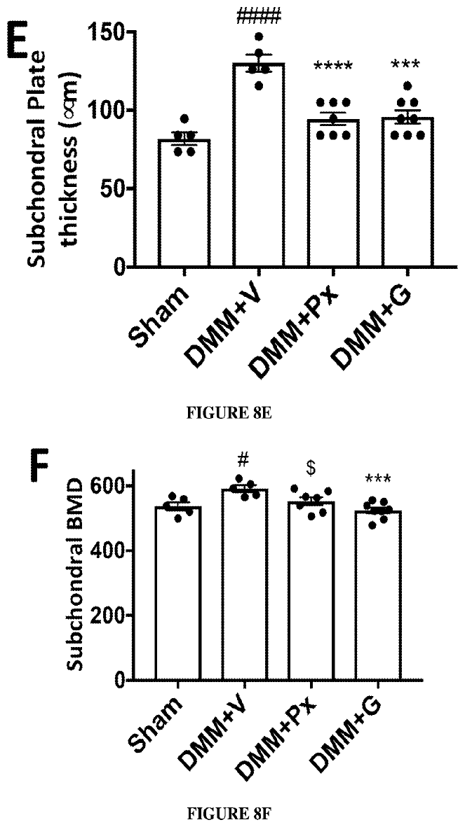

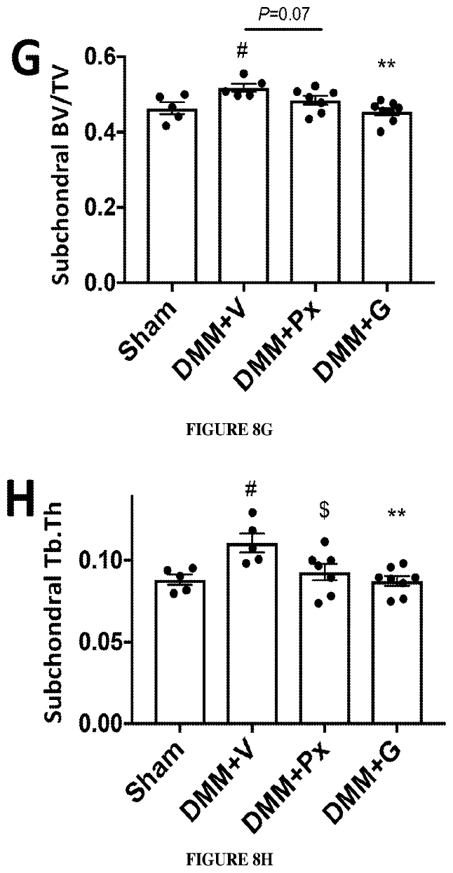

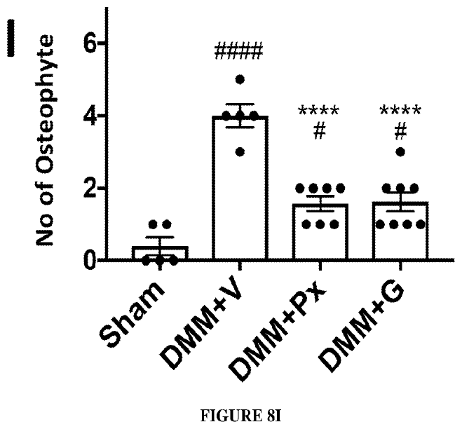

Paroxetine-mediated GRK2 inhibition prevents joint mineralization, subchondral bone remodeling and osteophyte formation in DMM mice. Micro-computed tomography (Micro-CT) 3D joint reconstructions of the knee joints of sham and vehicle-treated DMM mice showed that vehicle treated DMM mice exhibit increased joint mineralization particularly at the medial side, uneven bone surface of the tibia and the femur, and narrowing of the joint space (FIG. 8A), reflected in significantly increased knee bone volume (BV) (FIG. 8B) and knee total volume (TV) (FIG. 8C). All these pathological changes were ameliorated in paroxetine- and gallein-treated mice (FIG. 8A-C). Micro-CT analysis of the subchondral region demonstrated that, as compared to sham mice, vehicle-treated DMM mice showed significantly higher subchondral plate thickness (FIG. 8D, E), subchondral bone mineral density (BMD) (FIG. 8F), subchondral bone volume density (BV/TV) (FIG. 8G), subchondral trabecular thickness (Tb.Th) (FIG. 8H), and osteophyte number (FIG. 8I). Importantly, all these OA-associated subchondral abnormalities were alleviated by paroxetine and gallein treatment (FIG. 8 E-I). These effects might be secondary to improved OA or due to inhibition of pathological GRK2 signaling in the subchondral bone.

Discussion:

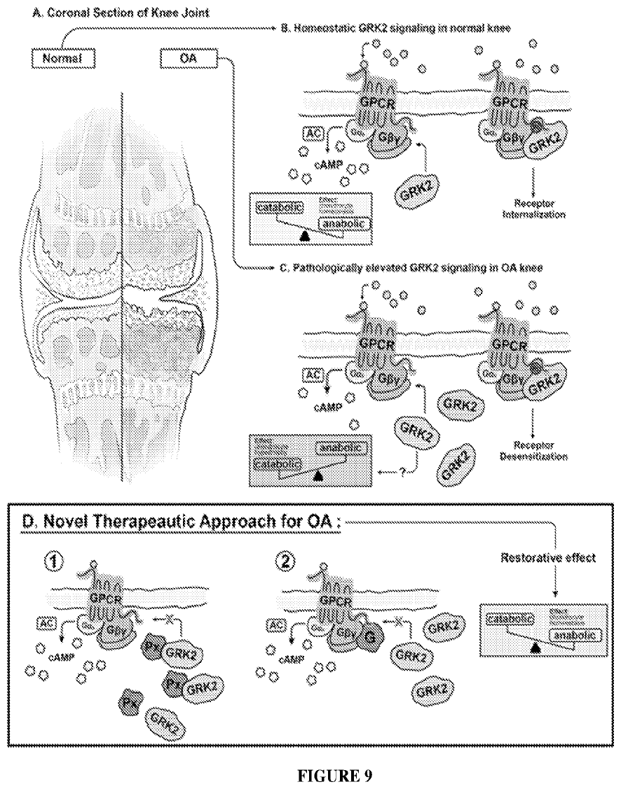

The data we present here demonstrate, for the first time, that the clinically used antidepressant “paroxetine” is a disease-modifying agent in OA that prevents cartilage degeneration and promotes matrix regeneration. The current study also establishes GRK2 as a major driver of OA progression, and GRK2 inhibition as a novel therapeutic approach for OA (FIG. 9).

Following DMM surgery, our data demonstrate a progressive increase in GRK2 expression that inversely correlates with cAMP levels over the course of the disease (FIG. 3), suggesting a relationship between GRK2 overexpression, GPCR desensitization and loss of Gas-cAMP signaling. GRK2-cKO in DMM mice conveyed significant chondro-protective and matrix-regenerative effects by recovering Gas activity and cAMP levels (FIG. 4). Importantly, recovered cAMP production in chondrocytes occurred as early as seven days following GRK2-cKO in DMM mice (FIG. 10), preceding any observable chondro-protective effects, which emphasizes the role of recovering Gas activity in inhibiting OA progression. These data are consistent with the reported role of Gas and cAMP in preventing CH (18, 19, 33-37) and promoting anabolic signaling in chondrocytes (38-40); as well as our previous findings in cardiovascular disease, where elevated GRK2 expression leads to GPCR desensitization and impaired Gas signaling (12, 41, 42). Altogether, we provide evidence for GRK2-mediated GPCR desensitization as a pathological driver of CH and cartilage degeneration in OA.

Paroxetine is an SSRI and a potent GRK2 inhibitor with higher selectivity for GRK2 over other GRKs both in vivo and in vitro (24, 43), Paroxetine-mediated inhibition of GRK2 prevents GPCR desensitization, exerting chondro-protective and matrix-regenerative effects in DMM mice (FIG. 5). Notably, inhibition of GRK2 may also exert GPCR-independent effects (44), whose impact on the homeostasis of chondrocytes and progression of OA cannot be excluded. However, our results from gallein, which prevents GPCR desensitization in a GRK2-independent way, show a therapeutic effect in DMM mice equivalent to that of paroxetine (FIG. 5). These data support the conclusion that paroxetine therapeutic effects in OA are exerted mainly through inhibition of GPCR desensitization. Importantly, paroxetine chondro-protective and matrix-regenerative effects are independent of its SSRI properties, since fluoxetine, another SSRI that possesses no GRK2 inhibitory effects, lacks these effects (FIG. 5).

Inflammation of the synovium, synovitis, is an important component of OA pathology (28-31). NSAIDS such as indomethacin are widely used to manage OA-associated pain and inflammation (25-27). Our data show that both paroxetine and gallein exert anti-inflammatory effects in OA mice equivalent to that of indomethacin, which is in agreement with the reported anti-inflammatory effects of both agents in different disease models (15, 45, 46). However, inhibition of inflammation is not sufficient to decelerate OA progression as evidenced in indomethacin-treated mice (FIG. 6), in consistence with clinical reports. Importantly, paroxetine anti-inflammatory effect is not secondary to decelerated OA, since the decelerated OA seen in chondrocyte specific GRK2-KO mice (FIG. 4) had no effect on synovial inflammation (FIG. 6). These data also indicate that GRK2 inhibition in chondrocytes is a central mechanism of paroxetine therapeutic effects in OA.

It is well established that microstructural changes in the subchondral bone environment are integral for OA-associated cartilage degeneration via cartilage-bone cross-talk (47-49), which was recently shown in DMM mice (50). Furthermore, osteophyte formation highly correlates with cartilage damage in OA (51). Consistent with this, our data show subchondral bone remodeling and increased osteophyte formation in DMM mice, which were dramatically normalized through GRK2 inhibition by paroxetine or gallein (FIG. 8). These results can be either secondary to paroxetine/gallein-mediated chondro-protection, or due to paroxetine/gallein-mediated inhibition of GRK2 in subchondral bone. The latter possibility suggests a direct role for GRK2 in OA-associated subchondral bone remodeling and osteophyte formation. Further studies employing conditional KO of GRK2 in the subchondral bone are required to dissect these mechanisms.

A limitation that faces a disease-modifying treatment for OA is the progressive degenerative nature of the disease, where extensive chondrocyte loss and cartilage degeneration in end stage OA are impossible to reverse or modify. In early stages of OA that follow a traumatic injury, chondrocytes enter a hypertrophic state as a type of compensatory mechanism to modify the extracellular matrix in response to increased load (49, 52). This pathological signaling starts at the site of injury and spreads throughout the articular surface leading to an almost full chondrocyte loss and complete degeneration of the articular surface (52). The ability of paroxetine or gallein to promote chondro-protective and anabolic signaling requires existing and viable chondrocytes that respond to treatment by regenerating their surrounding extracellular matrix. As a result, starting paroxetine or gallein treatment at end-stage OA may not have the same robust efficacy that we observe when treatment starts earlier, around mid-stage OA.

GRK2 is a ubiquitous GPCR kinase; thus, GRK2 inhibition recovers signaling of multiple families of GPCRs. Future studies analyzing the impact of paroxetine-mediated GRK2 inhibition on different GPCR signaling networks within articular chondrocytes will further our understanding of the mechanisms whereby paroxetine modifies OA. Also, further investigation of the optimum dosage and treatment regimen of paroxetine in various preclinical OA models is warranted to enable the clinical translation of this novel therapeutic approach.

Interdicting OA disease progression requires a holistic approach and understanding in order to attenuate pathological changes across multiple tissues in the joint. This study unveils a novel role for GRK2-mediated GPCR desensitization in promoting CH and cartilage degeneration, thus, accelerating OA progression (FIG. 9). Importantly, the present study is the first to show that GRK2 inhibition by paroxetine represents a novel approach that interdicts pathological changes across multiple tissues in a PTOA model, including articular cartilage, subchondral bone, and synovium. Of translational significance, attenuated CH as a result of paroxetine/gallein-mediated inhibition of GPCR desensitization was recapitulated in ex vivo cultured human OA cartilage (FIG. 7). As a clinically used antidepressant with known pharmacological and toxicological profiles, paroxetine represents a promising therapy for OA that can be easily translated from bench to bedside.

### REFERENCES

1. A. Litwic, M. H. Edwards, E. M. Dennison, C. Cooper, Epidemiology and burden of osteoarthritis. Br Med Bull 105, 185-199 (2013).
2. A. G. White, H. G. Birnbaum, C. Janagap, S. Buteau, J. Schein, Direct and indirect costs of pain therapy for osteoarthritis in an insured population in the United States. J Occup Environ Med 50, 998-1005 (2008).
3. V. Y. Ma, L. Chan, K. J. Carruthers, Incidence, prevalence, costs, and impact on disability of common conditions requiring rehabilitation in the United States: stroke, spinal cord injury, traumatic brain injury, multiple sclerosis, osteoarthritis, rheumatoid arthritis, limb loss, and back pain. Arch Phys Med Rehabil 95, 986-995 e981 (2014).
4. W. M. Hopman, M. B. Harrison, H. Coo, E. Friedberg, M. Buchanan, E. G. VanDenKerkhof, Associations between chronic disease, age and physical and mental health status. Chronic. Dis. Can. 29, 108-116 (2009).
5. L. B. Murphy, M. G. Cisternas, D. J. Pasta, C. G. Helmick, E. H. Yelin, Medical Expenditures and Earnings Losses Among US Adults With Arthritis in 2013. Arthritis Care Res (Hoboken) 70, 869-876 (2018).
6. A. A. Pitsillides, F. Beier, Cartilage biology in osteoarthritis—lessons from developmental biology. Nat Rev Rheumatol 7, 654-663 (2011).
7. P. M. van der Kraan, W. B. van den Berg, Chondrocyte hypertrophy and osteoarthritis: role in initiation and progression of cartilage degeneration? Osteoarthritis Cartilage 20, 223-232 (2012).
8. J. A. Hata, W. J. Koch, Phosphorylation of G protein-coupled receptors: GPCR kinases in heart disease. Molecular interventions 3, 264-272 (2003).
9. F. A. Kamal, J. G. Travers, B. C. Blaxall, G protein-coupled receptor kinases in cardiovascular disease: why “where” matters. Trends Cardiovasc Med 22, 213-219 (2012).
10. F. A. Kamal, A. V. Smrcka, B. C. Blaxall, Taking the heart failure battle inside the cell: small molecule targeting of Gbetagamma subunits. J Mol Cell Cardiol 51, 462-467 (2011).
11. F. A. Kamal, D. M. Mickelsen, K. M. Wegman, J. G. Travers, J. Moalem, S. R. Hammes, A. V. Smrcka, B. C. Blaxall, Simultaneous adrenal and cardiac GPCR-Gbeta gamma inhibition halts heart failure progression. Journal of the American College of Cardiology, (2014).
12. F. A. Kamal, J. G. Travers, A. E. Schafer, Q. Ma, P. Devaraj an, B. C. Blaxall, G Protein-Coupled Receptor-G-Protein betagamma-Subunit Signaling Mediates Renal Dysfunction and Fibrosis in Heart Failure. J Am Soc Nephrol, (2016).
13. C. Murga, A. C. Arcones, M. Cruces-Sande, A. M. Briones, M. Salaices, F. Mayor, Jr., G Protein-Coupled Receptor Kinase 2 (GRK2) as a Potential Therapeutic Target in Cardiovascular and Metabolic Diseases. Front Pharmacol 10, 112 (2019).
14. D. M. Thal, K. T. Homan, J. Chen, E. K. Wu, P. M. Hinkle, Z. M. Huang, J. K. Chuprun, J. Song, E. Gao, J. Y. Cheung, L. A. Sklar, W. J. Koch, J. J. Tesmer, Paroxetine is a direct inhibitor of g protein-coupled receptor kinase 2 and increases myocardial contractility. ACS Chem Biol 7, 1830-1839 (2012).

15. D. M. Lehmann, A. M. Seneviratne, A. V. Smrcka, Small molecule disruption of G protein beta gamma subunit signaling inhibits neutrophil chemotaxis and inflammation. Mol Pharmacol 73, 410-418 (2008).
16. J. G. Travers, F. A. Kamal, J. Robbins, K. E. Yutzey, B. C. Blaxall, Cardiac fibrosis: the fibroblast awakens. J Circulation Research 118, 1021-1040 (2016).
17. S. M. Schumacher, E. Gao, W. Zhu, X. Chen, J. K. Chuprun, A. M. Feldman, J. J. Tesmer, W. J. Koch, Paroxetine-mediated GRK2 inhibition reverses cardiac dysfunction and remodeling after myocardial infarction. Sci Transl Med 7, 277ra231 (2015).
18. A. S. Chagin, H. M. Kronenberg, Role of G-proteins in the differentiation of epiphyseal chondrocytes. J Mol Endocrinol 53, R39-45 (2014).
19. M. Bastepe, L. S. Weinstein, N. Ogata, H. Kawaguchi, H. Juppner, H. M. Kronenberg, U. I. Chung, Stimulatory G protein directly regulates hypertrophic differentiation of growth plate cartilage in vivo. Proc Natl Acad Sci USA 101, 14794-14799 (2004).
20. K. L. Culley, C. L. Dragomir, J. Chang, E. B. Wondimu, J. Coico, D. A. Plumb, M. Otero, M. B. Goldring, Mouse models of osteoarthritis: surgical model of posttraumatic osteoarthritis induced by destabilization of the medial meniscus. Methods Mol Biol 1226, 143-173 (2015).
21. S. S. Glasson, T. J. Blanchet, E. A. Morris, The surgical destabilization of the medial meniscus (DM_M) model of osteoarthritis in the 129/SvEv mouse. Osteoarthritis. Cartilage. 15, 1061-1069 (2007).
22. J. Lorenz, S. Grassel, Experimental osteoarthritis models in mice. Methods Mol Biol 1194, 401-419 (2014).
23. S. P. Henry, C. W. Jang, J. M. Deng, Z. Zhang, R. R. Behringer, B. de Crombrugghe, Generation of aggrecan-CreERT2 knockin mice for inducible Cre activity in adult cartilage. Genesis 47, 805-814 (2009).
24. D. M. Thal, K. T. Homan, J. Chen, E. K. Wu, P. M. Hinkle, Z. M. Huang, J. K. Chuprun, J. Song, E. Gao, J. Y. Cheung, Paroxetine is a direct inhibitor of g protein-coupled receptor kinase 2 and increases myocardial contractility. J ACS Chemical Biology 7, 1830-1839 (2012).
25. E. C. Huskisson, H. Berry, P. Gishen, R. Jubb, J. Whitehead, Effects of antiinflammatory drugs on the progression of osteoarthritis of the knee. LINK Study Group. Longitudinal Investigation of Nonsteroidal Antiinflammatory Drugs in Knee Osteoarthritis. The Journal of Rheumatology 22, 1941-1946 (1995).
26. J. Martel-Pelletier, A. J. Barr, F. M. Cicuttini, P. G. Conaghan, C. Cooper, M. B. Goldring, S. R. Goldring, G. Jones, A. J. Teichtahl, J.-P. Pelletier, Osteoarthritis. Nature Reviews Disease Primers 2, 16072 (2016).
27. T. Neogi, The epidemiology and impact of pain in osteoarthritis. J Osteoarthritis and Cartilage 21, 1145-1153 (2013).
28. C. R. Scanzello, S. R. Goldring, The role of synovitis in osteoarthritis pathogenesis. Bone 51, 249-257 (2012).
29. J. Sellam, F. Berenbaum, The role of synovitis in pathophysiology and clinical symptoms of osteoarthritis. J Nature Reviews Rheumatology 6, 625 (2010).
30. B. J. De Lange-Brokaar, A. Ioan-Facsinay, G. J. Van Osch, A.-M. Zuurmond, J. Schoones, R. E. Toes, T. W. Huizinga, M. Kloppenburg, Synovial inflammation, immune cells and their cytokines in osteoarthritis: a review. J Osteoarthritis and Cartilage 20, 1484-1499 (2012).
31. H. Zhang, C. Lin, C. Zeng, Z. Wang, H. Wang, J. Lu, X. Liu, Y. Shao, C. Zhao, J. Pan, S. Xu, Y. Zhang, D. Xie, D. Cai, X. Bai, Synovial macrophage M1 polarisation exacerbates experimental osteoarthritis partially through R-spondin-2. Annals of the rheumatic diseases 77, 1524-1534 (2018).
32. K. Miyamoto, B. Ohkawara, M. Ito, A. Masuda, A. Hirakawa, T. Sakai, H. Hiraiwa, T. Hamada, N. Ishiguro, K. Ohno, Fluoxetine ameliorates cartilage degradation in osteoarthritis by inhibiting Wnt/beta-catenin signaling. PloS one 12, e0184388 (2017).
33. A. S. Chagin, K. K. Vuppalapati, T. Kobayashi, J. Guo, T. Hirai, M. Chen, S. Offermanns, L. S. Weinstein, H. M. Kronenberg, G-protein stimulatory subunit alpha and Gq/11alpha G-proteins are both required to maintain quiescent stem-like chondrocytes. Nat Commun 5, 3673 (2014).
34. J. Guo, U. I. Chung, H. Kondo, F. R. Bringhurst, H. M. Kronenberg, The PTHIPTHrP receptor can delay chondrocyte hypertrophy in vivo without activating phospholipase C. Dev Cell 3, 183-194 (2002).
35. L. Wu, S. Zhang, R. Shkhyan, S. Lee, F. Gullo, C. D. Eliasberg, F. A. Petrigliano, K. Ba, J. Wang, Y. Lin, D. Evseenko, Kappa opioid receptor signaling protects cartilage tissue against posttraumatic degeneration. JCI Insight 2, e88553 (2017).
36. Y. Jin, Q. Cong, J. Gvozdenovic-Jeremic, J. Hu, Y. Zhang, R. Terkeltaub, Y. Yang, Enppl inhibits ectopic joint calcification and maintains articular chondrocytes by repressing hedgehog signaling. Development 145, (2018).
37. F. Yano, T. Saito, N. Ogata, T. Yamazawa, M. Iino, U. I. Chung, H. Kawaguchi, beta-catenin regulates parathyroid hormone/parathyroid hormone-related protein receptor signals and chondrocyte hypertrophy through binding to the intracellular C-terminal region of the receptor. Arthritis Rheum 65, 429-435 (2013).
38. J. M. Kim, J. S. Choi, Y. H. Kim, S. H. Jin, S. Lim, H. J. Jang, K. T. Kim, S. H. Ryu, P. G. Suh, An activator of the cAMP/PKA/CREB pathway promotes osteogenesis from human mesenchymal stem cells. J Cell Physiol 228, 617-626 (2013).
39. M. Quintero, G. Colantuoni, A. M. Khatib, A. Panasyuk, A. Lomri, D. R. Mitrovic, Granulocyte-macrophage colony stimulating factor activates proteoglycan, type II collagen, and cAMP production by rat articular chondrocytes through specific binding sites. J Rheumatol 28, 2075-2084 (2001).
40. J. B. Fitzgerald, M. Jin, D. Dean, D. J. Wood, M. H. Zheng, A. J. Grodzinsky, Mechanical compression of cartilage explants induces multiple time-dependent gene expression patterns and involves intracellular calcium and cyclic AMP. The Journal of biological chemistry 279, 19502-19511 (2004).
41. F. A. Kamal, D. M. Mickelsen, K. M. Wegman, J. G. Travers, J. Moalem, S. R. Hammes, A. V. Smrcka, B. C. Blaxall, Simultaneous adrenal and cardiac g-protein-coupled receptor-gbetagamma inhibition halts heart failure progression. Journal of the American College of Cardiology 63, 2549-2557 (2014).
42. J. G. Travers, F. A. Kamal, I. Valiente-Alandi, M. L. Nieman, M. A. Sargent, J. N. Lorenz, J. D. Molkentin, B. C. Blaxall, Pharmacological and Activated Fibroblast Targeting of Gbetagamma-GRK2 After Myocardial Ischemia Attenuates Heart Failure Progression. Journal of the American College of Cardiology 70, 958-971 (2017).
43. A. P. Campbell, A. V. Smrcka, Targeting G protein-coupled receptor signalling by blocking G proteins. Nature Reviews Drug Discovery 17, 789-803 (2018).
44. P. Penela, C. Murga, C. Ribas, V. Lafarga, F. Mayor, Jr., The complex G protein-coupled receptor kinase 2 (GRK2) interactome unveils new physiopathological targets. Br J Pharmacol 160, 821-832 (2010).
45. C. Han, Y Li, Y. Wang, D. Cui, T. Luo, Y. Zhang, Y Ma, W. Wei, Development of Inflammatory Immune Response-Related Drugs Based on G Protein-Coupled Receptor Kinase 2. Cellular physiology and biochemistry: international journal of experimental cellular physiology, biochemistry, and pharmacology 51, 729-745 (2018).
46. Q. Wang, L. Wang, L. Wu, M. Zhang, S. Hu, R. Wang, Y Han, Y. Wu, L. Zhang, X. Wang, W. Sun, W. Wei, Paroxetine alleviates T lymphocyte activation and infiltration to joints of collagen-induced arthritis. Sci Rep 7, 45364 (2017).
47. S. R. Goldring, M. B. Goldring, Changes in the osteochondral unit during osteoarthritis: structure, function and cartilage-bone crosstalk. J Nature Reviews Rheumatology 12, 632 (2016).
48. R. J. Lories, F. P. Luyten, The bone-cartilage unit in osteoarthritis. Nature Reviews Rheumatology 7, 43 (2011).
49. D. B. Burr, M. A. Gallant, Bone remodelling in osteoarthritis. Nature Reviews Rheumatology 8, 665 (2012).
50. P. Das Neves Borges, A. E. Forte, T. L. Vincent, D. Dini, M. Marenzana, Rapid, automated imaging of mouse articular cartilage by microCT for early detection of osteoarthritis and finite element modelling of joint mechanics. Osteoarthritis Cartilage 22, 1419-1428 (2014).
51. J. P. Pelletier, D. V. Jovanovic, V. Lascau-Coman, J. C. Fernandes, P. T. Manning, J. R. Connor, M. G. Currie, J. Martel-Pelletier, Selective inhibition of inducible nitric oxide synthase reduces progression of experimental osteoarthritis in vivo: possible link with the reduction in chondrocyte apoptosis and caspase 3 level. Arthritis Rheum 43, 1290-1299 (2000).
52. P. Van der Kraan, W. Van den Berg, Chondrocyte hypertrophy and osteoarthritis: role in initiation and progression of cartilage degeneration? J Osteoarthritis and Cartilage 20, 223-232 (2012).
53. H. K. Le Bleu, F. A. Kamal, M. Kelly, J. P. Ketz, M. J. Zuscik, R. A. Elbarbary, Extraction of high-quality RNA from human articular cartilage. Anal Biochem 518, 134-138 (2017).

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein; however, other combinations of steps, elements, components, and constituents are included, even though not explicitly stated.

1. A pharmaceutical composition comprising paroxetine or a pharmaceutically acceptable salt or derivative thereof; and one or more pharmaceutical acceptable carriers;
wherein the compound is present in an effective amount to increase PTH/PTH1R-mediated cAMP production by at least 2 fold.

2. The pharmaceutical composition of claim 1, wherein the PTH/PTH1R-mediated cAMP production is increased by inhibiting G-protein coupled receptor kinase 2 (GRK2).

3. The pharmaceutical composition of claim 1, wherein the composition further comprises parathyroid hormone (PTH).

4. A method of increasing PTH/PTH1R-mediated cAMP production in a subject in need thereof, the method comprising administering a paroxetine or a pharmaceutically acceptable salt or derivative thereof to the subject.

5. (canceled)

6. A method of treating an inflammatory disorder in a subject in need thereof, the method comprising administering a paroxetine or a pharmaceutically acceptable salt or derivative thereof to the subject.

7. (canceled)

8. The method of claim 4, wherein the paroxetine or a pharmaceutically acceptable salt or derivative thereof is present in a therapeutically effective amount to increase PTH/PTH1R-mediated cAMP production by at least 2 fold.

9. The method of claim 4, wherein the method comprises:
administering a pharmaceutical composition comprising paroxetine or a pharmaceutically acceptable salt or derivative thereof; and one or more pharmaceutical acceptable carriers;
wherein the compound is present in an effective amount to increase PTH/PTH1R-mediated cAMP production by at least 2 fold.

10. The method of claim 6, wherein the inflammatory disorder is arthritis.

11. The method of claim 6, wherein the inflammatory disorder is osteoarthritis.

12. The method of claim 4, wherein the subject is a human.

13. The method of claim 6, wherein the symptom is pain.

14. The method of claim 4, wherein the administration comprises local administration.

15. The method of claim 4, wherein the administration comprises a local delivery of the compounds incorporated within a gel, nanoparticles, microparticles, or an implant.
